(19) United States
(12) Patent Application Publication
(10) Pub. No.: US 2010/0030220 A1
(43) Pub. Date: Feb. 4, 2010

(54) BONE TREATMENT SYSTEMS AND METHODS

(75) Inventors: Csaba Truckai, Saratoga, CA (US); John H. Shadduck, Tiburon, CA (US)

Correspondence Address: KNOBBE MARTENS OLSON & BEAR LLP, 2040 MAIN STREET, FOURTEENTH FLOOR, IRVINE, CA, 92614, US

(73) Assignee: DFINE, INC., San Jose, CA (US)

(21) Appl. No.: 12/512,505

(22) Filed: Jul. 30, 2009

Related U.S. Application Data
(60) Provisional application No. 61/137,529, filed on Jul. 31, 2008.

(51) Int. Cl. A61B 17/58; A61N 1/39

(52) U.S. Cl. 606/93; 607/1

(57) ABSTRACT

Systems and methods for treating vertebral compression fractures are provided. In one embodiment, a bone cement injector system can include a first handle component that is detachably coupled to a second sleeve component having a distal end configured for positioning in bone, and a flow channel extending through the first and second components. The system can include a thermal energy emitter. The flow channel can have a flow channel surface with a material that that limits cement flow turbulence. At least a portion of the flow channel can have a non-round cross section.

### CROSS-REFERENCE TO RELATED APPLICATIONS

This application claims the benefit of U.S. Provisional Application No. 61/137,529, filed Jul. 31, 2008. This application is also related to the following U.S. patent application Ser. No. 11/209,035, filed Aug. 22, 2005, titled Bone Treatment Systems and Methods; Ser. No. 12/395,532, filed Feb. 27, 2009, titled Bone Treatment Systems and Methods; Provisional Application No. 60/842,805, filed Sep. 7, 2006, titled Bone Treatment Systems and Methods; No. 60/713,521, filed Sep. 1, 2005, titled Bone Treatment Systems and Methods; No. 60/929,936, filed Apr. 30, 2007, titled Bone Treatment Systems and Methods and No. 60/899,487, filed Feb. 5, 2007, titled Bone Treatment Systems and Methods. The entire contents of all of the above applications are hereby incorporated by reference and should be considered a part of this specification.

### BACKGROUND OF THE INVENTION

1. Field of the Invention

Embodiments of the present disclosure relate to bone cements and cement injection systems, and in some embodiments provide systems and methods for on-demand control of bone cement viscosity for treating vertebral compression fractures and for preventing cement extravasation, wherein a settable bone cement can comprise first and second cement precursors that are characterized by a post-mixing working interval in which viscosity changes at a low rate, for example, an extended interval in which the change of viscosity averages less than 50%/minute.

2. Description of the Related Art

Osteoporotic fractures are prevalent in the elderly, with an annual estimate of 1.5 million fractures in the United States alone. These include 750,000 vertebral compression fractures (VCFs) and 250,000 hip fractures. The annual cost of osteoporotic fractures in the United States has been estimated at $13.8 billion. The prevalence of VCFs in women age 50 and older has been estimated at 26%. The prevalence increases with age, reaching 40% among 80-year-old women. Medical advances aimed at slowing or arresting bone loss from aging have not provided solutions to this problem. Further, the population affected will grow steadily as life expectancy increases. Osteoporosis affects the entire skeleton but most commonly causes fractures in the spine and hip. Spinal or vertebral fractures also cause other serious side effects, with patients suffering from loss of height, deformity and persistent pain which can significantly impair mobility and quality of life. Fracture pain usually lasts 4 to 6 weeks, with intense pain at the fracture site. Chronic pain often occurs when one vertebral level is greatly collapsed or multiple levels are collapsed.

Postmenopausal women are predisposed to fractures, such as in the vertebrae, due to a decrease in bone mineral density that accompanies postmenopausal osteoporosis. Osteoporosis is a pathologic state that literally means “porous bones”. Skeletal bones are made up of a thick cortical shell and a strong inner meshwork, or cancellous bone, of collagen, calcium salts and other minerals. Cancellous bone is similar to a honeycomb, with blood vessels and bone marrow in the spaces. Osteoporosis describes a condition of decreased bone mass that leads to fragile bones which are at an increased risk for fractures. In an osteoporosis bone, the sponge-like cancellous bone has pores or voids that increase in dimension making the bone very fragile. In young, healthy bone tissue, bone breakdown occurs continually as the result of osteoclast activity, but the breakdown is balanced by new bone formation by osteoblasts. In an elderly patient, bone resorption can surpass bone formation thus resulting in deterioration of bone density. Osteoporosis occurs largely without symptoms until a fracture occurs.

Vertebroplasty and kyphoplasty are recently developed techniques for treating vertebral compression fractures. Percutaneous vertebroplasty was first reported by a French group in 1987 for the treatment of painful hemangiomas. In the 1990's, percutaneous vertebroplasty was extended to indications including osteoporotic vertebral compression fractures, traumatic compression fractures, and painful vertebral metastasis. Vertebroplasty is the percutaneous injection of PMMA (polymethylmethacrylate) into a fractured vertebral body via a trocar and cannula. The targeted vertebrae are identified under fluoroscopy. A needle is introduced into the vertebrae body under fluoroscopic control, to allow direct visualization. A bilateral transpedicular (through the pedicle of the vertebrae) approach is typical but the procedure can be done unilaterally. The bilateral transpedicular approach allows for more uniform PMMA infill of the vertebra.

In a bilateral approach, approximately 1 to 4 ml of PMMA or more is used on each side of the vertebra. Since the PMMA needs to be is forced into the cancellous bone, the techniques require high pressures and fairly low viscosity cement. Since the cortical bone of the targeted vertebra may have a recent fracture, there is the potential of PMMA leakage. The PMMA cement contains radiopaque materials so that when injected under live fluoroscopy, cement localization and leakage can be observed. The visualization of PMMA injection and extravasation are critical to the technique, as the physician generally terminates PMMA injection when leakage is observed. The cement is injected using syringes to allow the physician manual control of injection pressure.

Balloon kyphoplasty is a modification of percutaneous vertebroplasty. Balloon kyphoplasty involves a preliminary step comprising the percutaneous placement of an inflatable balloon tamp in the vertebral body. Inflation of the balloon creates a cavity in the bone prior to cement injection. In balloon kyphoplasty, the PMMA cement can be injected at a lower pressure into the collapsed vertebra since a cavity exists, when compared to conventional vertebroplasty. More recently, other forms of kyphoplasty have been developed in which various tools are used to create a pathway or cavity into which the bone cement is then injected.

The principal indications for any form of vertebroplasty are osteoporotic vertebral collapse with debilitating pain. Radiography and computed tomography must be performed in the days preceding treatment to determine the extent of vertebral collapse, the presence of epidural or foraminal stenosis caused by bone fragment retropulsion, the presence of cortical destruction or fracture and the visibility and degree of involvement of the pedicles.

Leakage of PMMA during vertebroplasty can result in very serious complications including compression of adjacent structures that necessitate emergency decompressive surgery. See Groen, R. et al., “Anatomical and Pathological Considerations in Percutaneous Vertebroplasty and Kyphoplasty: A Reappraisal of the Vertebral Venous System,” Spine Vol. 29, No. 13, pp 1465-1471, 2004. Leakage or extravasation of PMMA is a critical issue and can be divided into paravertebral leakage, venous infiltration, epidural leakage and intradiscal leakage. The exothermic reaction of PMMA carries potential catastrophic consequences if thermal damage were to extend to the dural sac, cord, and nerve roots. Surgical evacuation of leaked cement in the spinal canal has been reported. It has been found that leakage of PMMA is related to various clinical factors such as the vertebral compression pattern, and the extent of the cortical fracture, bone mineral density, the interval from injury to operation, the amount of PMMA injected and the location of the injector tip. In one recent study, close to 50% of vertebroplasty cases resulted in leakage of PMMA from the vertebral bodies. See Hyun-Woo Do et al., “The Analysis of Polymethylmethacrylate Leakage after Vertebroplasty for Vertebral Body Compression Fractures,” J. of Korean Neurosurg. Soc., Vol. 35, No. 5 (May 2004), pp 478-82, (http://www.jkns.or.kr/htm/abstract.asp?no=0042004086).

Another recent study was directed to the incidence of new VCFs adjacent to the vertebral bodies that were initially treated. Vertebroplasty patients often return with new pain caused by a new vertebral body fracture. Leakage of cement into an adjacent disc space during vertebroplasty increases the risk of a new fracture of adjacent vertebral bodies. See Am. J. Neuroradiol., February 2004; 25(2):175-80. The study found that 58% of vertebral bodies adjacent to a disc with cement leakage fractured during the follow-up period compared with 12% of vertebral bodies adjacent to a disc without cement leakage.

Another life-threatening complication of vertebroplasty is pulmonary embolism. See Bernhard, J. et al., “Asymptomatic Diffuse Pulmonary Embolism Caused by Acrylic Cement: An Unusual Complication of Percutaneous Vertebroplasty,” Ann. Rheum. Dis., 62:85-86, 2003. The vapors from PMMA preparation and injection also are cause for concern. See Kirby, B, et al., “Acute Bronchospasm Due to Exposure to Polymethylmethacrylate Vapors During Percutaneous Vertebroplasty,” Am. J. Roentgenol., 180:543-544, 2003.

### SUMMARY OF THE INVENTION

There is a general need to provide bone cements and methods for use in treatment of vertebral compression fractures that provide a greater degree of control over introduction of cement and that provide better outcomes. The present invention meets this need and provides several other advantages in a novel and nonobvious manner.

Certain embodiments provide bone cement injectors and control systems that allow for vertebroplasty procedures that inject cement having a substantially constant viscosity over an extended cement injection interval.

A computer controller can be provided to control cement flow parameters in the injector and energy delivery parameters for selectively accelerating polymerization of bone cement before the cement contacts the patient's body.

In some embodiments, a medical device for applying energy to a bone cement can comprise a member, at least one energy emitter and a bone cement source. The member can have a flow channel extending through the member. In some embodiments, at least a portion of the flow channel has a non-round cross section. The at least one energy emitter can be operatively coupled to the member and can be configured to apply energy to a bone cement flowing through the flow channel. The bone cement source can also be coupleable to the flow channel.

The medical device can further have an interior surface of the flow channel with a material that limits cement flow turbulence, in certain embodiments. The at least one energy emitter can take many forms, including at least one electrode, at least first and second opposing polarity electrodes, a resistive heater, a light source, and an ultrasound source.

In some embodiments of the medical device, the member can be made at least partly of a polymer. The polymer can be any of or none of the following: transparent, electrically insulative, electrically conductive, a positive temperature coefficient of resistance.

Certain embodiments of a medical device for applying energy to a bone cement can comprise a member with a flow channel extending therethrough, and at least one energy emitter operatively coupled to the member and configured to apply energy to bone cement in the flow channel. The medical device can further comprise a bone cement source coupleable to the flow channel. In some embodiments, an interior surface of the flow channel comprises a material that limits cement flow turbulence.

In still further embodiments of the medical device at least a portion of the flow channel can have a non-round cross section; this section may comprise the material that limits cement flow turbulence.

The material that limits cement flow turbulence can increase laminar flow and/or have a static coefficient of friction of less than 0.5. In some embodiments, the material is selected from the group comprising PTFE (polytetrafluoroethylene), PFA (perfluoroalkoxy), polyamide, polyvinyl chloride, FEP (fluorinatedethylenepropylene), ECTFE (ethylenechlorotrifluoroethylene), ETFE, polyethylene, PVDF and silicone.

These and other objects of the present invention will become readily apparent upon further review of the following drawings and specification.

### BRIEF DESCRIPTION OF THE DRAWINGS

In order to better understand the invention and to see how it may be carried out in practice, some preferred embodiments are next described, by way of non-limiting examples only, with reference to the accompanying drawings, in which like reference characters denote corresponding features consistently throughout similar embodiments in the attached drawings.

FIG. 1 is a schematic, perspective view of a bone cement injection system in accordance with one embodiment.

FIG. 2 is a schematic, exploded side view of the system of FIG. 1 illustrating some of the bone cement injection components de-mated from one another.

FIG. 3 is a schematic illustration of one embodiment of a thermal emitter component of the system of FIGS. 1 and 2.

FIG. 4 is a schematic, exploded perspective view components of the system of FIGS. 1-2 in combination with an embodiment of a force application and amplification system, a pressurization mechanism and in communication with an energy source and a controller.

FIG. 5 is an enlarged, assembly view of an embodiment of a force application and amplification system and a pressurization mechanism of the system of FIG. 4.

FIG. 6 is a perspective view of some of the components of the system of FIGS. 1-5 with a perspective view of an embodiment of an energy source and controller.

FIG. 7 is chart indicating certain time-viscosity curves for PMMA bone cements.

FIG. 8A is diagram indicating a method of utilizing applied energy and an energy-delivery algorithm to accelerate the polymerization of a PMMA bone cement to provide a selected time-viscosity curve.

FIG. 8B is a chart indicating a modified time-viscosity curve for a PMMA bone cement of FIG. 7 when modified by applied energy from a thermal energy emitter and a selected energy-delivery algorithm according to an embodiment of the present disclosure .

FIGS. 8C and 8D are images of PMMA bone cement exiting an injector. FIG. 8C is PMMA bone cement exiting the injector without applied energy and FIG. 8D is the same PMMA bone cement exiting an injector as modified by applied energy according to one embodiment of energy-delivery algorithm.

FIG. 9 is chart indicating another modified time-viscosity curve for the PMMA bone cement of FIG. 7 and 8A when modified by applied energy using an alternative energy-delivery algorithm.

FIG. 10 is a chart indicating time-viscosity curves for an embodiment of PMMA bone cement as in FIG. 8A at different ambient temperatures.

FIG. 11 is a view of another embodiment of a bone cement injection system with some components de-mated from one another wherein the system includes first and second thermal energy emitters.

FIG. 12 is a plot illustrating setting time as a function of the concentration of BPO and DMPT present within embodiments of a bone cement composition.

FIG. 13 is a plot illustrating the temperature-time behavior of embodiments of a bone cement composition under conditions where the composition is and is not heated.

FIG. 14 is a plot illustrating the viscosity-time behavior of embodiments of a bone cement composition heated to temperatures ranging between about 25° C. to 55° C.

FIG. 15 is a chart indicating time-viscosity curves for two embodiments of PMMA bone cement of this disclosure as well as other commercially available PMMA bone cements.

FIG. 16 is perspective view of a handle portion of a bone cement injection system in accordance with some embodiments.

FIG. 17 is a cut-away view of the handle portion of FIG. 16 showing an energy emitter of a heating system for applying energy to a flow of bone cement.

FIG. 18 is an enlarged sectional view of the energy emitter of FIG. 17.

FIG. 19 is another sectional view of the energy emitter of FIGS. 17 and 18.

### DETAILED DESCRIPTION OF THE PREFERRED EMBODIMENTS

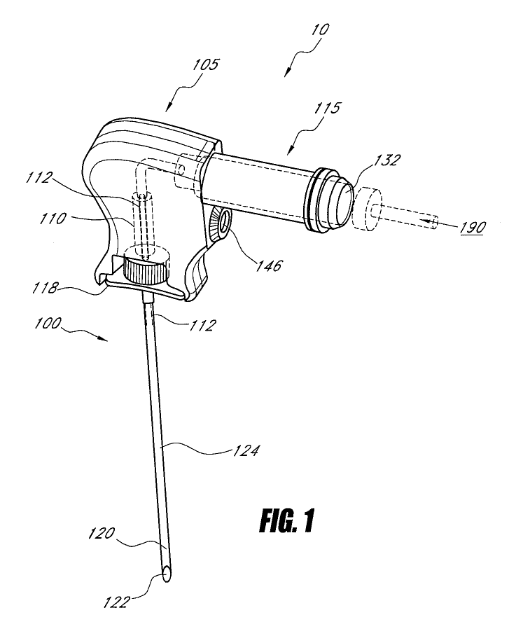

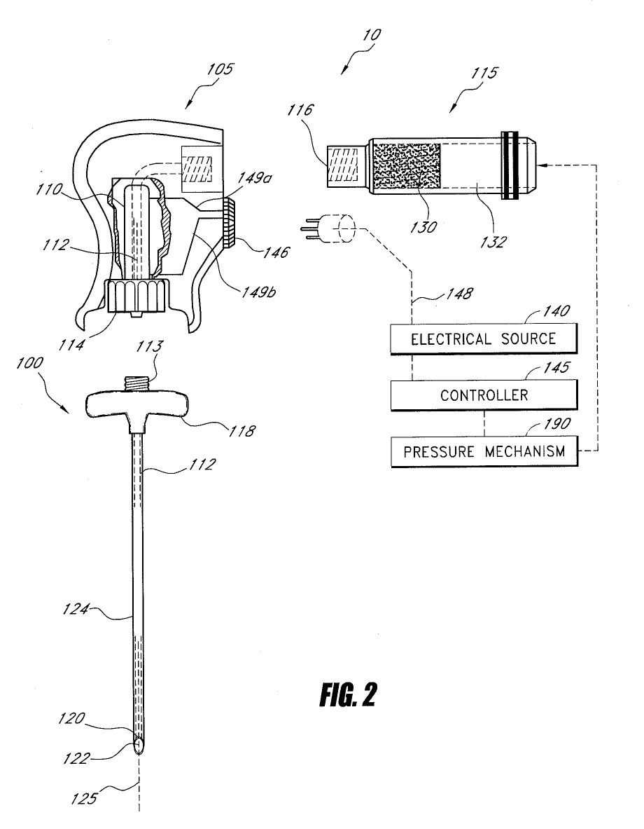

For purposes of understanding the principles of the invention, reference will now be made to the embodiments illustrated in the drawings and accompanying text. As background, a vertebroplasty procedure using embodiments of the present disclosure may introduce the injector of FIGS. 1-2 through a pedicle of a vertebra, or in a parapedicular approach, for accessing the osteoporotic cancellous bone. The initial aspects of the procedure can be similar to a conventional percutaneous vertebroplasty wherein the patient is placed in a prone position on an operating table. The patient is typically under conscious sedation, although general anesthesia is an alternative. The physician injects a local anesthetic (e.g., 1% Lidocaine) into the region overlying the targeted pedicle or pedicles as well as the periosteum of the pedicle(s). Thereafter, the physician may use a scalpel to make a 1 to 5 mm skin incision over each targeted pedicle. Thereafter, a bone cement injector can be advanced through the pedicle into the anterior region of the vertebral body, which typically is the region of greatest compression and fracture. The physician confirms the introducer path posterior to the pedicle, through the pedicle and within the vertebral body by anteroposterior and lateral X-Ray projection fluoroscopic views or by other methods. The introduction of infill material as described below can be imaged several times, or continuously, during the treatment depending on the imaging method.

### DEFINITIONS

The terms “bone cement,” “bone fill,” “bone fill material,” “infill material,” and “infill composition” include their ordinary meaning as known to those skilled in the art and may include any material for infilling a bone that includes an in-situ hardenable or settable cement and compositions that can be infused with such a hardenable cement. The fill material also can include other fillers, such as filaments, microspheres, powders, granular elements, flakes, chips, tubules and the like, autograft or allograft materials, as well as other chemicals, pharmacological agents or other bioactive agents.

The term “flowable material” includes its ordinary meaning as known to those skilled in the art and may include a material continuum that is unable to withstand any static shear stress and responds with a substantially irrecoverable flow (e.g., a fluid), unlike an elastic material or elastomer that responds to shear stress with a recoverable deformation. Flowable materials may include fill material or composites that may include a first, fluid component alone or in combination with a second, elastic, or inelastic material component that responds to stress with a flow, no matter the proportions of the first and second component. It may be understood that the above shear test does not apply to the second component alone.

The terms “substantially” or “substantial” include their ordinary meaning as known to those skilled in the art and may mean largely but not entirely. For example, “substantially” and “substantial” may mean about 50% to about 99.999%, about 80% to about 99.999% or about 90% to about 99.999%.

The term “vertebroplasty” includes its ordinary meaning as known to those skilled in the art and may include any procedure where fill material is delivered into the interior of a vertebra.

The term “osteoplasty” includes its ordinary meaning as known to those skilled in the art and may include any procedure where fill material is delivered into the interior of a bone.

In FIG. 1, an embodiment of a system 10 is shown that includes a first component or bone cement injector 100 which may extend into cancellous bone of a vertebra, and a second component or cement activation component 105 which includes an emitter 110 for applying energy to bone cement. The first and second components 100 and 105 may include a flow passageway or channel 112 extending therethrough for delivering flowable bone cement into a bone. The bone cement injector component 100 and the cement activation component 105 can be integrated into a unitary device or can be de-matable as shown in FIG. 2 by a mechanism such as a threaded portion 113 and rotatable screw-on fitting 114. Other configurations are also possible. As can be seen in FIGS. 1 and 2, a source of bone cement in the form of a syringe-type body 115 can also couple to the system, for example, by way of a threaded fitting 116.

Referring to FIG. 2, the bone cement injector 100 has a proximal end 118 and a distal end 120 with at least one flow outlet 122 therein to direct a flow of cement into a bone. The extension portion 124 of the injector 100 can be made of any suitable metal or plastic sleeve with flow channel 112 extending therethrough to the flow outlet 122. The flow outlet 122 may be present as a side port to direct cement flow transverse relative to the axis 125 of extension portion 124 or, alternatively, can be positioned at the distal termination of extension portion 124 in order to direct cement flows distally. In another embodiment (not shown) the extension portion 124 can include first and second concentric sleeves that can be positioned so as to be rotated relative to one another to align or misalign respective first and second flow outlets to allow selectively directed cement flows to be more or less axial relative to axis 125 of extension portion 124.

Now turning to the cut-away view of FIG. 2, it can be seen that second component 105 includes a handle portion that carries an emitter 110 for applying thermal energy to a cement flow within the flow channel 112 that extends through the emitter 110. As will be described further below, the emitter 110 may apply thermal energy to bone cement 130 delivered from chamber 132 of source 115 to flow through the emitter 110 to therein cause the viscosity of the cement to increase to a selected, higher viscosity value as the cement exits the injector flow outlet 122 into bone. The controlled application of energy to bone cement 130 may enable the physician to select a setting rate for the cement to reach a selected polymerization endpoint as the cement is being introduced into the vertebra, for example, allowing a high viscosity that will prevent unwanted cement extravasation.

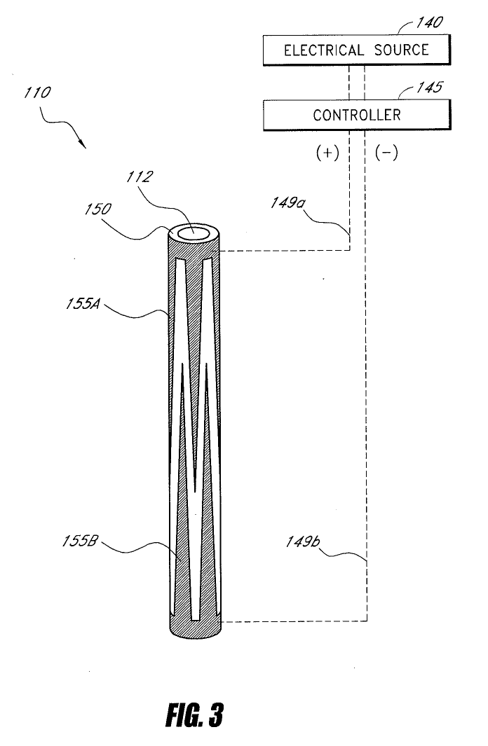

Referring to FIGS. 2 and 3, in one embodiment, the thermal energy emitter 110 may be coupled to an electrical source 140 and controller 145 by an electrical connector 146 and a cable 148. In FIG. 2, it can be seen that electrical leads 149a and 149b may be coupled with connector 146 and extend to the emitter 110. As can be seen in FIG. 3, one embodiment of thermal energy emitter 110 has a wall portion 150 that includes a polymeric positive temperature coefficient of resistance (PTCR) material with spaced apart interlaced surface electrodes 155A and 155B as described in Provisional Application No. 60/907,469 filed Apr. 3, 2007 titled Bone Treatment Systems and Methods. In this embodiment, the thermal emitter 110 and wall 150 thereof may resistively heat to thereby cause controlled thermal effects in bone cement 130 flowing therethrough. It may be appreciated that FIG. 3 is a schematic representation of one embodiment of thermal energy emitter 110 which can have any elongated or truncated shape or geometry, tapered or non-tapered form, or include the wall of a collapsible thin-wall element. Further, the positive (+) and negative (−) polarity electrodes 155A and 155B can have any spaced apart arrangement, for example radially spaced apart, helically spaced apart, axially spaced apart or any combination thereof. This resistively heated PTCR material of the emitter 110 may further generate a signal that indicates flow rate as described in Provisional Application No. 60/907,469 which in turn can be utilized by controller 145 to modulate energy applied to the bone cement therein, and/or modulate the flow rate of cement 130 which can be driven by a motor or stored energy mechanism. In another embodiment, the emitter can be any non-PTCR resistive heater such as a resistive coil heater.

In other embodiments, the thermal energy emitter 110 can include a PTCR constant temperature heater as described above or may include one or more of a resistive heater, a fiber optic emitter, a light channel, an ultrasound transducer, an electrode and an antenna. Accordingly in any such embodiment, the energy source 140 can include at least one of a voltage source, a radiofrequency source, an electromagnetic energy source, a non-coherent light source, a laser source, an LED source, a microwave source, a magnetic source and an ultrasound source that is operatively coupled to the emitter 110.

Referring FIG. 2, it can be understood that a pressure mechanism 190 is coupleable to a bone cement source or a syringe 115 for driving the bone cement 130 through the system 10. The pressure mechanism 190 can include any suitable manual drive system or an automated drive system such as any pump, screw drive, pneumatic drive, hydraulic drive, cable drive or the like. Such automated drive systems maybe coupled to controller 145 to modulate the flow rate of cement through the system.

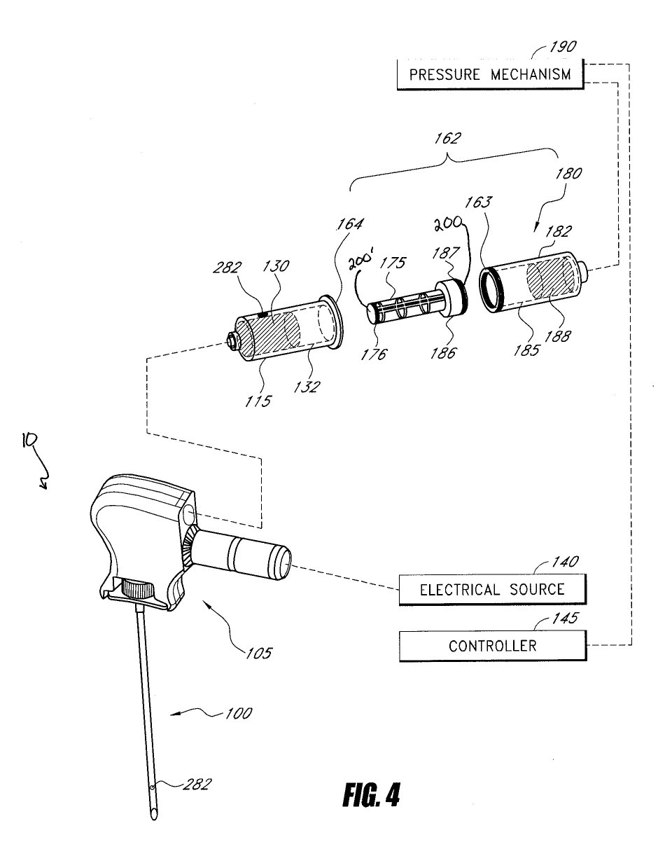

In one embodiment shown in FIGS. 4-6, the system 10 may further include a hydraulic system 162 with a fitting 163 that may detachably couple to fitting 164 of the bone cement source 115. In this embodiment, the bone cement source 115 may include a syringe body with cement-carrying bore or chamber 132 that carries a pre-polymerized, partially polymerized or recently-mixed bone cement 130 therein. The hydraulic system 162 may further include a rigid plunger or actuator member 175 with o-ring or rubber head 176 that may move in chamber 132 so as to push the cement 130 through the syringe chamber 132 and the flow channel 112 in the system 100.

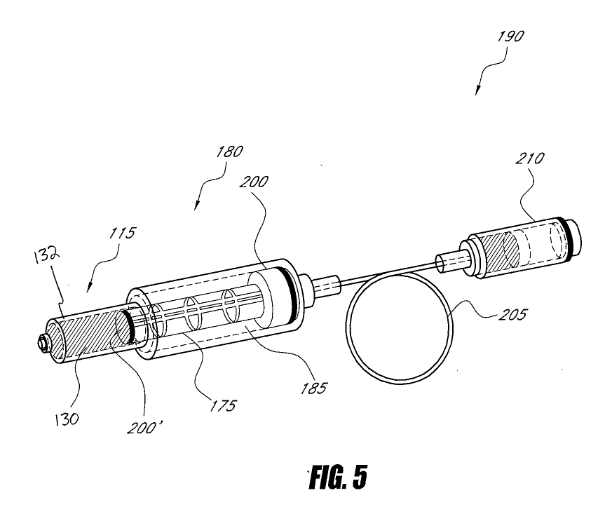

Still referring to FIGS. 4-6, a force application and amplification component 180 of the hydraulic system 162 may reversibly couple to the bone cement source 115, where the force application and amplification component 180 includes a body 182 with pressurizable bore or chamber 185 therein that slidably receives the proximal end 186 of an actuator member 175. The proximal end 186 of actuator member 175 may include an o-ring or gasket 187 so that the bore 185 can be pressurized with flow media 188 by the pressure source 190 in order to drive the actuator member 175 distally to thereby displace bone cement 130 from the chamber 132 in the cement source or syringe 115. In one embodiment, illustrated in FIG. 5, the surface area of an interface 200 between the actuator member 175 and pressurized flow media 188 may be larger than the surface area of an interface 200′ between the actuator member 175 and the bone cement 130 so as to thereby provide pressure amplification between the pressurizable chamber 185 and chamber 132 of the cement source or syringe. In one embodiment as indicated in FIGS. 4 and 5, the surface area of interface 200 may be at least about 150% of the surface area of interface 200′, at least about 200% of the surface area of interface 200′, at least about 250% of the surface area of interface 200′ and at least about 300% of the surface area of interface 200′.

Referring to FIGS. 4 and 5, in one embodiment, a force application and amplification component 188 may be employed in the following manner. In a first operation, a bone fill material injector with a displaceable, non-fluid actuator component intermediate a first fluid chamber and a second cement or fill-carrying chamber may be provided. In a second operation, a flow of media may be provided into the first fluid chamber at a first pressure to thereby displace the actuator component to impinge on and eject bone cement or fill at a higher second pressure from the second chamber into a vertebra. In a non-limiting example, a second pressure may be provided in the cement-carrying chamber 132 that is greater than the first pressure in the pressurizable chamber 185.

In one embodiment, the second pressure may be at least about 50% higher than the first pressure in the pressurizable chamber 185. In another embodiment, the second pressure may be at least about 75% higher than the first pressure in the pressurizable chamber 185. In another embodiment, the second pressure may be at least about 100% higher than the first pressure in the pressurizable chamber 185. In another embodiment, the second pressure may be at least about 200% higher than the first pressure in the pressurizable chamber 185. In another embodiment, the second pressure may be at least about 300% higher that the first pressure in the pressurizable chamber 185.

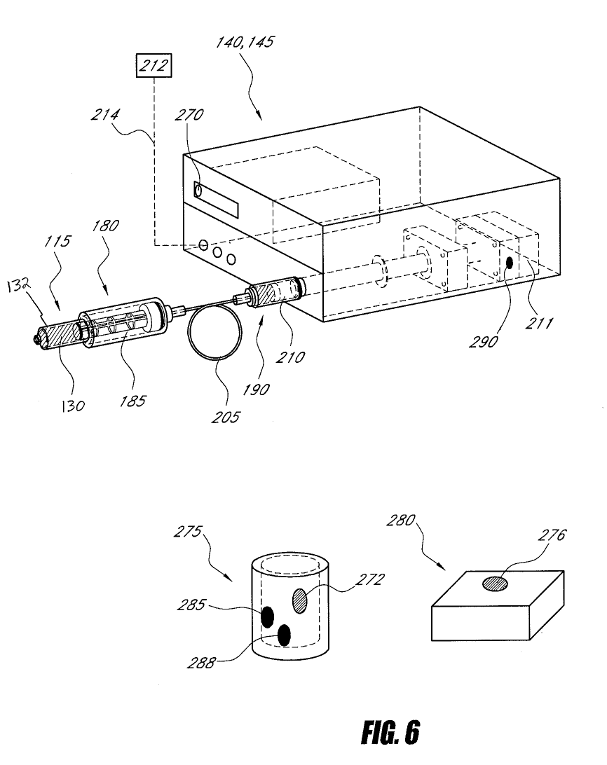

Referring to FIGS. 5 and 6, one embodiment of pressurizing mechanism for providing pressure to the force application and amplification component 180 may include a pneumatic or hydraulic line 205 that extends to pressure mechanism 190 such as a syringe pump 210, which is manually driven or motor-driven as is known in the art. In one embodiment, as shown in FIG. 6, the syringe pump 210 may be driven by an electric motor 211 operatively coupled to controller 145 to allow modulation of the pressure or driving force in combination with the control of energy delivery by emitter 110 from energy source 140.

It may be appreciated that the pressurizing mechanism or pressure source 210 can include any type of mechanism or pump known in the art to actuate the actuator member 175 to move the bone cement in chamber 132. For example, a suitable mechanism can include a piezoelectric element for pumping fluid, an ultrasonic pump element, a compressed air system for creating pressure, a compressed gas cartridge for creating pressure, an electromagnetic pump for creating pressure, an air-hammer system for creating pressure, a mechanism for capturing forces from a phase change in a fluid media, a spring mechanism that may releaseably store energy, a spring mechanism and a ratchet, a fluid flow system and a valve, a screw pump, a peristaltic pump, a diaphragm pump, rotodynamic pumps, positive displacement pumps, and combinations thereof.

Referring to FIG. 6, another feature of embodiments of the present disclosure is a remote switch 212 for actuating the pressure mechanism 190. In one embodiment, a cable 214 extends from the controller 145 so that the physician can stand outside of the radiation field created by any imaging system used while treating a vertebra or other bone treatment site. In another embodiment, the switch 212 can be wirelessly connected to the system as is known in the art. In another embodiment (not shown), an elongated cable 214 and switch 212 can be directly coupled to the injector or other component of the system 10.

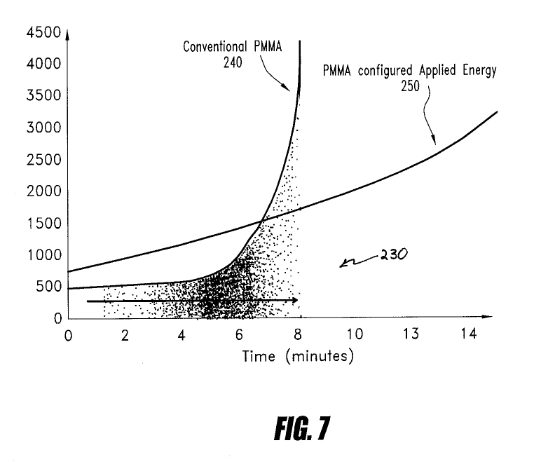

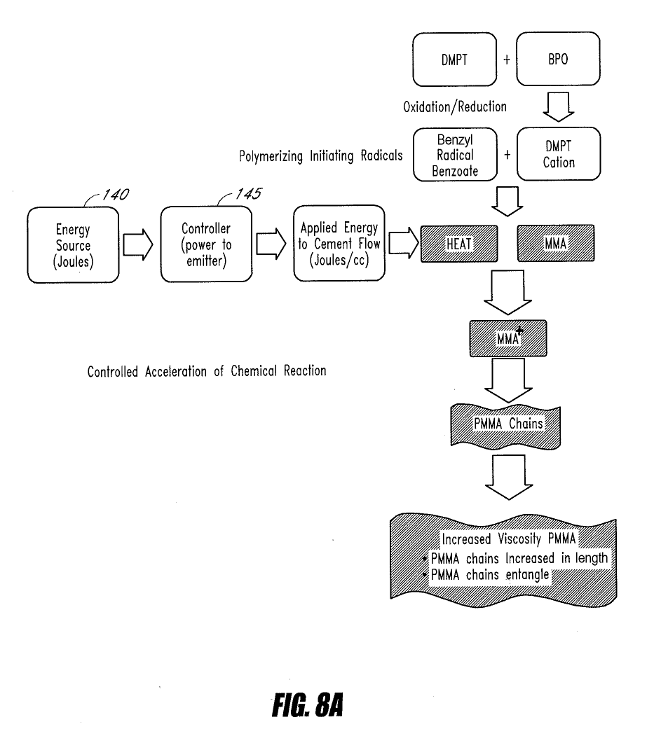

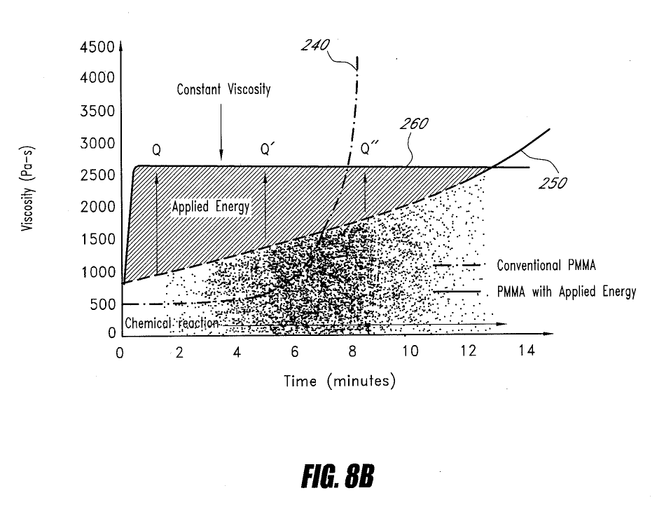

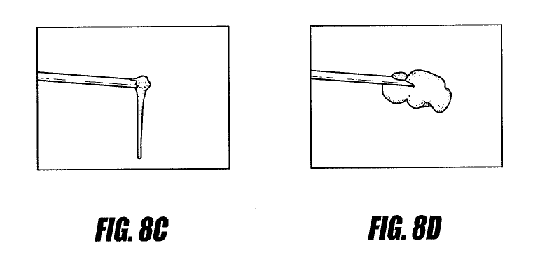

Now turning to FIGS. 7, 8A and 8B, the figures illustrate certain embodiments of a method wherein controlled application of energy to a bone cement 130 can provide a bone cement with a controlled, on-demand increased viscosity and a controlled set time compared to a prior art bone cement. FIG. 7 depicts a prior art bone cement known in the art, such as a PMMA bone cement, that has a time-viscosity curve 240 where the cement substantially hardens or cures within about 8 to 10 minutes post-mixing. On the horizontal axis of FIGS. 7, and 8B, the time point zero indicates the time at which the mixing of bone cement precursors, such as monomer and polymer components, is approximately completed.

As can be seen in time viscosity curve 240 for the prior art bone cement, the cement increases in viscosity from about 500 Pa·s to about 750 Pa·s from time zero to about 6 minutes post-mixing. Thereafter, the viscosity of the prior art cement increases very rapidly over the time interval from 6 minutes to 8 minutes post-mixing to a viscosity greater than 4000 Pa·s. A prior art bone cement having the time-viscosity curve 240 in FIG. 7 may be considered to have a fairly high viscosity for injection in the range of 500 Pa·s. At this viscosity range, however, the bone cement can still have flow characteristics that result in extravasation.

Still referring to FIG. 7, it can be understood that the curing reaction of the bone cement involves an exothermic chemical reaction that initiates a polymerization process that is dictated, at least in part, by the composition of the bone cement precursors, such as one or more of a PMMA polymer, monomer and initiator. FIG. 7 indicates at 230 the exothermic curing reaction over time as a gradation where the lighter gradation region indicates a lesser degree of chemical reaction and heat—and the darker gradation region indicates a greater degree of chemical reaction and heat leading to more rapid polymerization of the bone cement precursors.

Now turning to FIG. 8A, the block diagram illustrates an embodiment of a method of utilizing applied energy and an energy-delivery algorithm to accelerate the polymerization of a PMMA bone cement to provide a selected time-viscosity curve as in FIG. 8B. In FIGS. 7 and 8B, it can be seen that the time-viscosity curve 250 of one embodiment of a bone cement can have an initial viscosity is in the range of about 750 Pa·s at time zero post-mixing and thereafter the viscosity increases in a more linear manner over 10 to 14 minutes post-mixing than the prior art bone cements depicted with curve 240. This embodiment of bone cement that can provide a time-viscosity curve 250 as in FIGS. 7 and 8B, may include a PMMA cement composition as described in U.S. Provisional Application No. 60/899,487 filed on Feb. 5, 2007, titled Bone Treatment Systems and Methods, and U.S. application Ser. No. 12/024,969, filed on Feb. 5, 2008, titled Bone Treatment Systems and Methods, which are incorporated herein by this reference in their entirety. As can be seen in FIG. 8B, the bone cement 130, or more particularly, the mixing of the cement precursors includes a first curing reaction source for curing the bone cement as described above and can result in the predetermined exothermic curing reaction post-mixing that is indicated by the gradations of reaction under the time-viscosity curve 250.

Still referring to FIG. 8B, the chart illustrates a PMMA bone cement with time-viscosity curve 250 together with a modified time-viscosity curve 260. The modified time-viscosity curve may be provided by the application of energy employing an embodiment of the system 10 as depicted in FIGS. 1 and 4-6. In other words, FIG. 8B illustrates one embodiment where the bone cement 130 undergoes a curing process (i.e., the time-viscosity curve 250) owing to self-heating of the composition as components of the bone cement composition react with each other. This curing process may be further influenced be by the applied energy from energy source 140, controller 145 and emitter 110 to provide the modified time-viscosity curve 260 for cement injection into a bone in order to prevent extravasation.

As can be understood from FIG. 8B, the modulation of applied energy over time from the second curing source or emitter 110, indicated schematically at energy applications Q, Q′ and Q″, can be provided to complement the thermal energy generated by the exothermic reaction of the bone cement components in order to provide a substantially constant cement viscosity over a selected working time. This aspect of embodiments of the present disclosure allows, for the first time, the provision of bone cements having a controlled and substantially constant, viscosity that is selected so as to inhibit extravasation.

The bone cement 130 and system 10 of embodiments of the present disclosure are therefore notable in that a typical treatment of a vertebral compression fracture (VCF) requires cement injection over a period of several minutes, for example from about 2 to 10 minutes or about 2 to 6 minutes, or about 2 to 4 minutes. The physician typically injects a small amount of bone cement, for example about 1 to 2 cc, then pauses cement injection for the purpose of imaging the injected cement to check for extravasation, then injects additional cement and then images, etc. The steps of injecting and imaging may be repeated from 2 to 10 times or more, wherein the complete treatment interval can take 4 to 6 minutes or more. It can be easily understood that a cement with a working time of at least 5-6 minutes is needed for a typical treatment of a VCF—otherwise the first batch of cement may be too advanced in the curing process (see curve 240 in FIG. 7) and a second batch of cement may need to be mixed. In embodiments of the cement 130 and system 10, however, as indicated in FIG. 8B at 260, the cement viscosity can be approximately constant, thus providing a very long working time of about 8-10 minutes or more.

It should be appreciated that, in the chart of FIG. 8B, the contribution to bone cement curing owing to self-heating of the bone cement composition and applied energy are indicated by shaded areas below curves 250 and 260. This graphic representation, however, is for conceptual purposes only, as the vertical axis measures viscosity in Pa·s. The actual applied energy, indicated at Q to Q″, may be determined by analysis of the actual polymerization reaction time of a selected bone cement composition at a selected ambient temperature and atmospheric pressure.

Thus, in one embodiment, the bone cement system includes: a first energy source and a second energy source, different from one another that facilitate a curing reaction occurring within a bone cement. The first energy source includes heat generated by an exothermic curing reaction resulting from mixture of bone cement precursor components. The second energy source includes thermal energy introduced into the bone cement by a thermal energy emitter 110 that may provide a selected amount of energy to the bone cement. The system further includes a controller 145 that may modulate the thermal energy provided to the bone cement composition by the thermal energy emitter 110. In this manner, the curing reaction of the bone cement composition may be controlled over a selected working time. It can be understood from U.S. Provisional Application No. 60/899,487 and U.S. application Ser. No. 12/024,969, that PMMA cement compositions can be created to provide highly-extended working times.

Such bone cements in combination with the system 10 can thus allow for selected working times of at least about 6 minutes, about 8 minutes, about 10 minutes, about 12 minutes, about 14 minutes, about 16 minutes, about 18 minutes, about 20 minutes, and about 25 minutes. Further, the disclosure provides a control system that allows for providing a bone cement within a selected cement viscosity range as it exits the injector outlet 122 over the selected working time. Further, the disclosure provides a controller that is capable of providing a substantially constant cement viscosity over the selected working time. Further, the disclosure provides a controller that is capable of providing a plurality of selected time-viscosity profiles of the cement as it exits the injector.

In one embodiment, the bone cement system may include: a first and second energy source for causing a controlled curing reaction in a bone cement. The first energy source may include an exothermic curing reaction which occurs in response to mixing cement precursor compositions. The second energy source may include a thermal energy emitter capable of applying energy to bone cement in order to vary an exothermic curing reaction of the bone cement. The system may further include a controller capable of modulating the applied energy from the emitter to thereby control the exothermic curing reaction over a selected working time. The controller may be capable of modulating applied energy to provide a selected bone cement viscosity over a working time of at least about 2 minutes, at least about 4 minutes, at least about 6 minutes, at least about 8 minutes, at least about 10 minutes, at least about 12 minutes, at least about 14 minutes, at least about 16 minutes, at least about 18 minutes, at least about 20 minutes, and at least about 25 minutes.

In further embodiments the control system 10 may allow for application of energy to a bone cement so as to provide a bone cement that possesses a selected cement viscosity range as it exits the injector outlet 122 over the selected working time. In certain embodiments, the selected viscosity range may include, but is not limited to, about 600 Pa·s, about 800 Pa·s, about 1000 Pa·s, about 1200 Pa·s, about 1400 Pa·s, about 1600 Pa·s, about 1800 Pa·s, about 2000 Pa·s, about 2500 Pa·s, about 3000 Pa·s and about 4000 Pa·s.

In another embodiment, a method of preparing a curable bone cement for injection into a vertebra may be provided. The method can include: mixing bone cement precursors such that post-mixing provides a first non-variable curing reaction in the bone cement; and applying energy to the bone cement from an external source to provide a second variable curing reaction in the bone cement, wherein applied energy from the external source is controlled by a controller to permit a combination non-variable and variable curing reaction thereby providing a selected cement viscosity.

Embodiments of the method may further include varying the amount of energy applied from the external source in response to a selected length of a post-mixing interval. Further embodiments of the method can include varying the applied energy from the external source in response to ambient temperature that is measured by a temperature sensor in the system.

Further, embodiments of the method can include varying the applied energy from the external source in response to a selected injection rate of the bone cement flow through the system. Embodiments of the method can include varying the applied energy from the external source to provide a bone cement having an injection viscosity of at least about 500 Pa·s, at least about 1000 Pa·s, at least about 1500 Pa·s, at least about 2000 Pa·s, at least about 3000 Pa·s and at least about 4000 Pa·s.

In further embodiments, a method of involves preparing a curable bone cement for injection into a vertebra may be provided which allows a bone cement to exhibit a selected time-viscosity profile. The method can include: mixing bone cement precursors so as to cause a curing reaction characterized by a first time-viscosity profile of the bone cement, actuating an energy controller to controllably apply energy to the bone cement from an external source so as to cause the bone cement to adopt a second time-viscosity profile, different from the first time-viscosity profile, and injecting the cement characterized by the second time-viscosity profile into the vertebra. In embodiments of this method, the cement viscosity may be at least about 500 Pa·s, at least about 1000 Pa·s, at least about 1500 Pa·s, at least about 2000 Pa·s, at least about 3000 Pa·s or at least about 4000 Pa·s. Embodiments of the method can also include actuating the controller to modulate applied energy in response to control signals including, but not limited to, the length of a cement post-mixing interval, the ambient temperature, the bone cement temperature and the rate of bone cement injection into the vertebra.

As may be understood from FIG. 8B and the description above, some embodiments allow for cement injection at a viscosity range of over about 1500 Pa·s, about 2000 Pa·s or about 2500 Pa·s which have been found to be beneficial for substantially preventing extravasation of the cement. In one embodiment, the bone treatment system comprises: first and second energy sources for causing a controlled curing reaction in a bone cement, wherein the first energy source includes a predetermined exothermic curing reaction in response to mixing cement precursor compositions, and the second energy source includes a thermal energy emitter configured for providing a variable curing reaction, and a controller configured for modulating applied energy from the emitter to thereby control the curing reaction over a selected working time. The controller can be capable of modulating applied energy to provide a selected cement viscosity over a working time of at least about 2 minutes, at least about 4 minutes, at least about 6 minutes, at least about 8 minutes, at least about 10 minutes, at least about 12 minutes, at least about 14 minutes, at least about 16 minutes, at least about 18 minutes, at least about 20 minutes, at least about 25 minutes.

In another embodiment, a bone treatment system 10 may be provided that employs algorithms for modulating energy applied to the bone cement 130. The bone treatment system 10 can include a bone cement injector system, a thermal energy emitter 110 that may deliver energy to a flow of bone cement through the injector system, and a controller. The controller 145 may include hardware and/or software for implementing one or more algorithms for modulating applied energy from the emitter to a bone cement flow. The energy-delivery algorithms may be further employed to increase applied energy from about zero to a selected value at a rate that inhibits vaporization of at least a portion of a monomer portion of the bone cement 130.

In another embodiment of the present disclosure, the controller 145 enable a physician to select a particular approximate cement viscosity using a selector mechanism operatively connected to the controller 145. The selector mechanism, in some embodiments, is on the controller 145 and can comprise a button, switch, interface, etc. In one embodiment, the physician can select among a plurality of substantially constant viscosities that can be delivered over the working time. Examples of ranges of such viscosities may include less that about 1,000 Pa·s and greater than about 1,500 Pa·s. It should be appreciated that, in certain embodiments, two to six or more selections may be enabled by the controller 145, with each selection being a viscosity range useful for a particular purpose, such as about 1,000 Pa·s for treating more dense bone when extravasation is of a lesser concern, or between about 4,000 Pa·s and 6,000 Pa·s in a treatment of a vertebral fracture to prevent extravasation and to apply forces to vertebral endplates to reduce the fracture.

The benefits of such viscosity control may be observed in FIGS. 8C and 8D, which, respectively, are images of a PMMA bone cement exiting an injector without applied energy and the same PMMA bone cement exiting an injector as modified by applied energy according to one embodiment of energy-delivery algorithm. The bone cement emerging from the injector without the benefit of applied energy is of relatively low viscosity, as evidenced by the ease with which the bone cement is deformed by the force of gravity. Such behavior indicates the bone cement of FIG. 8C may be prone to extravasation. In contrast, the bone cement modified by applied energy of high viscosity, as evidenced by its accumulation about the end of the injector. Such behavior indicates that the bone cement of FIG. 8D is not prone to extravasation.

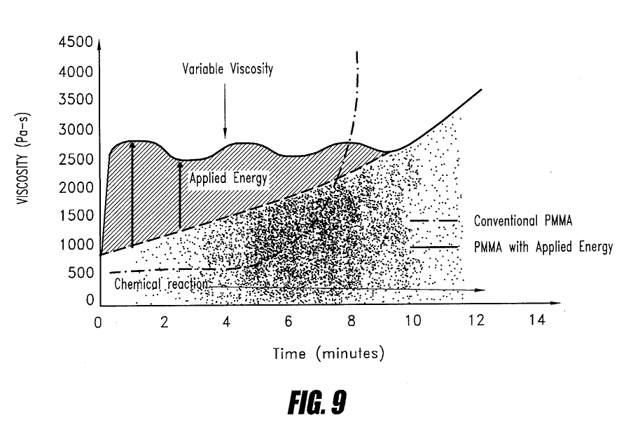

In another embodiment, referring to FIG. 9, the controller 145 may also allow the physician to select an energy-delivery algorithm in the controller 145 to provide a variable viscosity. For example, an algorithm could provide increases and decreases in cement viscosity as the cement exits the injector following the application of energy to the cement flow. Beneficially, such algorithms may provide substantially automated control of the application of energy to the composition by the system 10.

In another aspect of the time-viscosity curve 260 depicted in FIG. 8B, the system and method can provide a controller 145 and energy emitter 110 that can apply energy sufficient to very rapidly increase the viscosity of bone cement to a selected viscosity that will not allow for extravasation. As can be seen in FIG. 8B, the time-viscosity curve 260 within 15-30 seconds can be elevated to above 2000 Pa·s. A method of bone cement treatment can encompass utilizing an energy emitter 110 that applies energy to bone cement to controllably increase its viscosity in less than 2 minutes or less than 1 minute by at least 200 Pa·s, at least 500 Pa·s or at least 1,000 Pa·s. Alternatively, embodiments of a method of bone cement treatment may include utilizing an energy emitter that applies energy to bone cement to controllably increase the viscosity in less than 2 minutes or less than 1 minute to at least 1,000 Pa·s, at least 1,500 Pa·s, at least 2,000 Pa·s or at least 2,500 Pa·s.

In another embodiment, a bone treatment system may include a bone cement injector system that includes a thermal energy emitter 110 that may deliver energy to a bone cement within the injector system, a controller 145 that may modulate applied energy from the emitter to control a curing reaction of the cement, and a sensor system operatively coupled to the injector system for measuring an operational parameter of bone cement 130 within the system. In FIG. 6, in one embodiment, it can be seen that one sensor of the sensor system may include a temperature sensor, indicated at 270 which is disposed in controller assembly 140. The temperature sensor 270 in the controller assembly can allow for input into the system control algorithms for modulating applied energy from emitter 110 dependent on ambient air temperature in the operating room environment. Such control algorithms may be of significant utility, as the ambient temperature of an operating room may affect the time-viscosity curve of an exothermic PMMA-based bone cement.

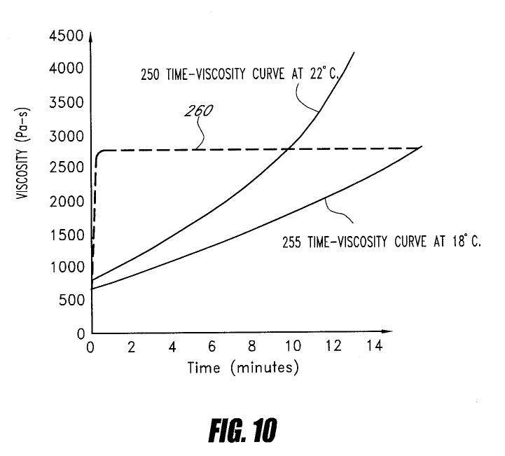

FIG. 10 provides a schematic graphical representation of the time-viscosity response, 250 and 255, respectively, of an embodiment of the bone cement of FIG. 8A after mixing at ambient temperatures of about 22° C. and 18° C. It can be seen that different levels energy may be applied to achieve a similar time-viscosity curve 260 of FIG. 10. For example, less energy may be applied to bone cement at about 22° C. than is applied to the bone cement at about 18° C. in order to achieve the time-viscosity response 160, as the higher temperature bone cement, prior to energy application, contains more energy than lower temperature bone cement. Thus, in an embodiment, a method may include providing inputs for the control algorithms for controlling applied energy to cement flows that factor in ambient temperatures. In some embodiments other temperatures can be measured and factored into the control algorithms in addition to or instead of the ambient temperature.

In order to facilitate energy application to the bone cement composition in a repeatable manner, the system 10 may further include a temperature sensor 272 disposed in a mixing device or assembly 275 (see FIG. 6). The mixing assembly 275 may include any container that receives bone cement precursors for mixing before placement of the mixed cement in the bone cement source 115. In certain embodiments, the temperature sensor 272 may be placed in the cement mixing assembly 275 because cement may be stored in a hospital in an environment having a lower or higher temperature than the operating room which may affect the time-viscosity curve of the cement. The temperature sensor 272 can be operatively coupled to the controller by a cable or a wireless transmitter system. In certain embodiments, the sensor 272 may be unitary with the mixing assembly 275 and disposable. In alternative embodiments, the sensor 272 can be reusable and detachable from the mixing assembly 275.

In another embodiment, still referring to FIG. 6, a temperature sensor 276 may be operatively connected to one or more packages 280 of the bone cement precursors to thereby indicate the actual temperature of the cement precursor(s) prior to mixing. Such a temperature sensor 276 may indicate the stored temperature and/or the length of time that such cement precursors have been in the operating room when compared to ambient room temperature measured by sensor 270 in the controller 145. This sensor 276 can include one or more temperature sensors that may include, but are not limited to, thermocouples, or thermochromic inks. The temperature sensors 276 may be further disposed on the surface of the bone cement package 280, allowing for visual identification of the temperature of the cement precursors. In this manner, a doctor or technician may read the temperature of the package 280 and manually input this temperature into the controller 145 to enable automatic adjustment of the energy delivery algorithms. In another embodiment, referring back to FIG. 4, at least one temperature sensor 282 can be located in cement source 115 of the system and/or in a distal portion of the injector component 100 for monitoring cement temperature in a cement flow within the system 10.

In another embodiment, the bone cement system 10 and more particularly the cement mixing assembly 275 of FIG. 6 may include a sensor, switch or indication mechanism 285 for indicating an approximate time of initiation of bone cement mixing. Such a sensor or indication mechanism 285 can include any manually-actuated mechanism coupled to the controller or a mechanism that senses the disposition of the cement precursors in the mixing assembly or the actuation of any moveable mixing component of the assembly, and combinations thereof. The system and controller 145 may in this manner provide one or more visual, aural and/or tactile signals indicating that a selected mixing time interval has been reached. This signal may enable consistent measurement of the time at which mixing of the bone cement is completed, also referred to as the zero post-mixing time, such that the viscosity at this time may be similar in all cases. Beneficially, by precise, consistent measurement of the zero post-mixing time, energy may be properly applied as described above. The system also can include a sensor, switch or indication mechanism 288 that can indicate the termination of bone cement mixing and thus time zero on a time-viscosity curve as in FIG. 9 which may be needed for setting the algorithms in the controller 145 for controlling applied energy and the cement flow rate.

In another embodiment, the bone cement system 10 may include a sensor that measures and indicates the bone cement flow rate within the flow passageway in the injector system. In the embodiment of FIG. 6, a motor drive system 211 can drive the cement via the hydraulic system at a substantially constant rate through the injector 100 and emitter 110. As shown, a sensor 290 is operatively coupled to the motor drive 211 which can measure the force being applied by the drive to cause the desired cement flow through the system, which can in turn be used to sense any tendency for a slow-down in the desired flow rate, for example due to an unanticipated increase in viscosity of the bone cement in the system 10. Upon such sensing, the controller 145 can increase the flow rate or decrease the applied energy from emitter 110 to allow a selected cement viscosity and flow rate from the injector 100 into bone to be maintained.

In one embodiment, the system 10 may be employed in order to provide the bone cement 130 with working time for polymerizing from an initial state to a selected endpoint of at least about: 10 minutes, 12 minutes, 14 minutes, 16 minutes, 18 minutes, 20 minutes, 25 minutes, 30 minutes and 40 minutes, as disclosed in Provisional Application No. 60/899,487. In an embodiment of the present disclosure, the initial state may include a first selected viscosity range of the bone cement 130 within about 90 to 600 seconds after completion of mixing of the bone cement components. In another embodiment of the disclosure, the selected endpoint of the bone cement 130 may include a second selected viscosity range that substantially inhibits bone cement extravasation. Herein, the terms “polymerization rate” and “working time” may be used alternatively to describe aspects of the time interval over which the cement polymerizes from the initial state to the selected endpoint.

As can be understood from FIGS. 1-6, the energy source 140 may also be capable of applying energy to the bone cement 130 via the emitter 110 and accelerating a polymerization rate of the bone cement by at least about: 20%, 30%, 40%, 50%, 60%, 70%, 80%, 90% and 95%, as compared to the polymerization rate achieved absent this application of energy. In another embodiment, the energy source 140 and controller 145 could be configured for accelerating the polymerization rate of the bone cement 130 to the selected endpoint in less than about: 1 second, 5 seconds, 10 seconds, 20 seconds, 30 seconds, 45 seconds, 60 seconds and 2 minutes.

An embodiment of a method of using the system 10 of FIGS. 1-6 to treat a vertebra is also provided. The method can include a first operation.of introducing a cement injector needle into a vertebra. The needle may include a flow channel extending from a proximal injector end to a distal injector end possessing a flow outlet. The method may further include a second operation of causing a flow of bone cement from the bone cement source through a flow channel in an energy-delivery component and the injector needle. The method may additionally include applying energy from the energy-delivery component to the flow of bone cement so as to cause a change in the setting rate of the cement so as to reach a selected polymerization endpoint. In this method, the applied energy may accelerate setting of a bone cement before it exits the flow outlet of the injector. The method and the selected polymerization endpoint can advantageously provide a viscosity that substantially prevents cement extravasation following introduction into the vertebra.

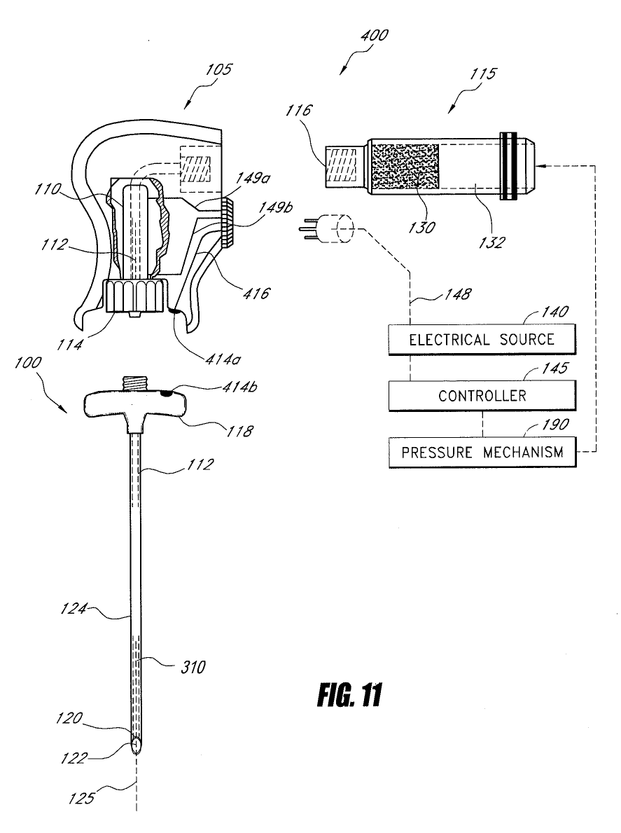

In another embodiment, referring to FIG. 11, a bone cement system 400 may include a first and a second thermal energy emitter for controlled application of energy to a bone cement flow within the flow passageway 112 of the injector system. More particularly, a first emitter 110 is shown disposed in the first handle component 105 as described previously. A second emitter 310 is shown disposed in a medial or distal portion of the second extension component 100 of the system. A controller 145 may be capable of modulating applied energy from the first and second emitters, 110 and 310, to provide a controlled curing reaction of the flow of bone cement. In one method of use, the first emitter 110 can apply energy to warm the flow of cement to accelerate polymerization so that the selected flow rate carries the cement to the location of the second emitter 310 at a viscosity of less than about 500 to 1000 Pa·s and thereafter the applied energy of the second emitter 310 may increase the viscosity to greater than about 2000 Pa·s. In this manner, the bone cement viscosity within the flow channel 112 can be kept at a level that can be pushed with a low level of pressure and the final viscosity of the bone cement exiting the outlet 122 can be at a relatively high viscosity, for example at a level capable of fracturing cancellous bone, such as greater than about 2000 Pa·s. It should be recognized that the viscosities given above are examples, the particular viscosity of the flow from the emitters 110 and 310 can depend on many factors, including the cement used, the treatment being performed, etc.

FIG. 11 further illustrates electrical connector components 414a and 414b provided in the interface between the first and second components, 100 and 105. These electrical connector components 414a, 414b can provide an electrical connection from electrical source 140 to the emitter 310 via electrical wires indicated at 416 in the handle portion 105 of the system. It may be appreciated that the second emitter 310 can be a PTCR emitter as described previously or any other type of heating element. The heating element can have any length including the entire length of the extension portion 124. In one embodiment, the emitter 110 in handle component 105 has a length of less than about 50 mm and can carry a volume of cement that is less than about: 1.0 cc, 0.8 cc, 0.6 cc, 0.4 and 0.2 cc.

In another embodiment of a method, the energy-delivery emitter 110 is actuated by the operator from a location outside any imaging field. The cable carrying an actuation switch can be any suitable length, for example about 10 to 15 feet in length.

Further embodiments of the present disclosure relate to bone cement compositions and formulations for use in the bone cement delivery systems described above. The bone cement formulations can provide for an extended working time, since the viscosity can be altered and increased on demand when injected.

Bone cements, such as polymethyl methacrylate (PMMA), have been used in orthopedic procedures for several decades, with initial use in the field of anchoring endoprostheses in a bone. An example of this includes skeletal joints such as in the hip replaced with a prosthetic joint. About one million joint replacement operations are performed each year in the U.S. Frequently, the prosthetic joint is cemented into the bone using an acrylic bone cement such as PMMA. In recent years, bone cements also have been widely used in vertebroplasty procedures wherein the cement is injected into a fractured vertebra to stabilize the fracture and eliminate micromotion that causes pain.

Polymethyl methacrylate bone cement, prior to injection, comprises a powder component and a liquid monomer component. The powder component comprises granules of methyl methacrylate or polymethyl methacrylate, an X-ray contrast agent and a radical initiator. Typically, barium sulfate or zirconium dioxide is used as an X-ray contrast agent. Benzoyl peroxide (BPO) is typically used as radical initiator. The liquid monomer component typically consists of liquid methyl methacrylate (MMI), an activator, such as N,N-dimethyl-p-toluidine (DMPT) and a stabilizer, such as hydroquinone (HQ). Prior to injecting PMMA bone cements, the powder component and the monomer component are mixed and thereafter the bone cement hardens within several minutes following radical polymerization of the monomer.

Typical bone cement formulations (including PMMA formulations) used for vertebroplasty have a fairly rapid cement curing time after mixing of the powder and liquid components. This allows the physician to spend less time waiting for the cement to increase in viscosity prior to injection. Further, the higher viscosity cement is less prone to unwanted extravasation which can cause serious complications. The disadvantage of such current formulations is that the “working time” of the cement is relatively short-for example about 5 to 8 minutes-in which the cement is within a selected viscosity range that allows for reasonably low injection pressures while still being fairly viscous to help limit cement extravasation. In some bone cement formulations, the viscosity ranges between approximately 50 to 500 N s/m² and is measured according to ASTM standard F451, “Standard Specification for Acrylic Bone Cement,” which is hereby incorporated by reference in its entirety.

In one embodiment, a bone cement of provides a formulation adapted for use with the cement injectors and energy delivery systems described above. These formulations are distinct from conventional formulations and have greatly extended working times for use in vertebroplasty procedures with the “on-demand” viscosity control methods and apparatus disclosed herein and in applications listed and incorporated by reference above.

In one embodiment, a bone cement provides a formulation adapted for injection into a patient's body, wherein the setting time is about 25 minutes or more, more preferably about 30 minutes or more, more preferably about 35 minutes or more, and even more preferably about 40 minutes or more. Setting time is measured in accordance with ASTM standard F451.

In one embodiment, a bone cement, prior to mixing and setting, comprises a powder component and a liquid component. The powder component comprises a PMMA that is about 64% to 75% by weight based on overall weight of the powder component. In this formulation, an X-ray contrast medium is about 27% to 32% by weight based on overall weight of the powder component. The X-ray contrast medium, in one embodiment, comprises barium sulfate (BaSO₄) or zirconium dioxide (ZrO₂). In one embodiment, the formulation further includes BPO that is about 0.4% to 0.8% by weight based on overall weight of the powder component. In another embodiment, the BPO is by weight based on overall weight of the powder component, less than 0.6%, 0.4% and 0.2%. In such formulations, the liquid component includes MMA that is greater than about 99% by weight based on overall weight of the liquid component. In such formulations, the liquid component includes DMPT that is less than about 1% by weight based on overall weight of the liquid component. In such formulations, the liquid component includes hydroquinone that ranges between about 30 and 120 ppm of the liquid component. In such formulations, the liquid weight/powder weight ratio is equal to or greater than about 0.4. In such formulations, the PMMA comprises particles having a mean diameter ranging from about 25 microns to 200 microns or ranging from about 50 microns to 100 microns.

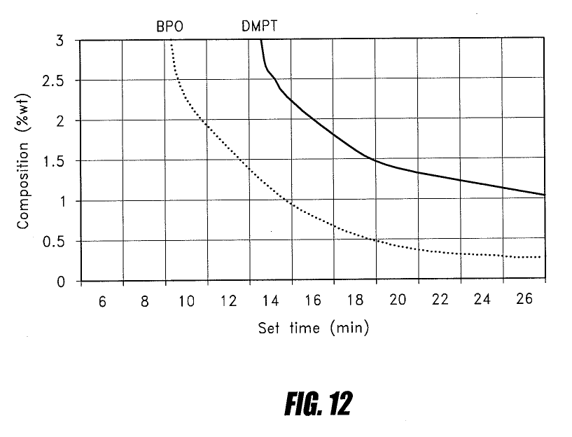

In certain embodiments, the concentrations of benzoyl peroxide and DMPT may be varied in order to adjust setting times. Studies examining the influence of bone cement concentration on setting times (FIG. 12) have demonstrated that, in bone cements comprising BPO and DMPT, increases in BPO and DMPT concentration increase the set time of the bone cement. The data further illustrate that, of the two bone cement constituents, BPO has a greater rate of effect on set time than does DMPT based on the percent weight. Thus, in certain embodiments of a bone cement composition, the concentration of BPO, DMPT, and combinations thereof, may be increased within the ranges discussed above so as to increase the setting time of the composition.

The setting time of the cement may also be influenced by applying energy to the bone cement composition. As discussed above, embodiments of the injector system of FIGS. 1-6 may be configured to deliver energy to the bone cement composition. In certain embodiments, the applied energy may heat the bone cement composition to a selected temperature.

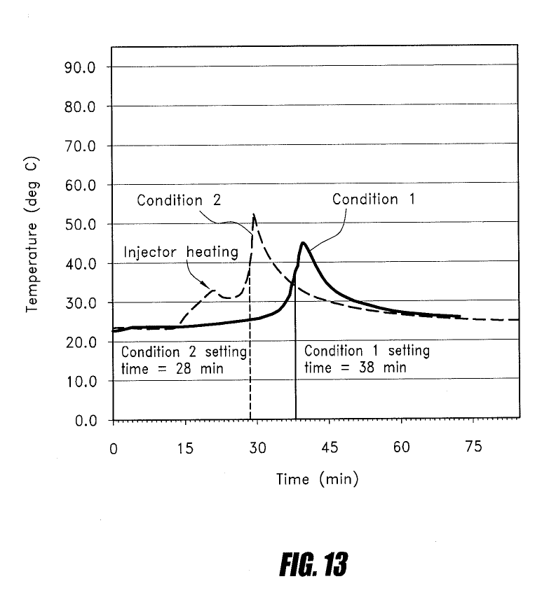

FIG. 13 illustrates temperature as a function of time from initial mixing for one embodiment of the bone composition so injected. The solid line of FIG. 13 represents the behavior of the composition when it is not heated by the injector system, referred to as condition 1. It is observed that, under condition 1, the composition exhibits three regimes. The first regime is low heating rate regime, where the temperature of the composition increases modestly with time. In this regime, the composition begins to slowly self-heat due the onset of a chemical reaction between at least a portion of its components. The second regime is a high heating rate regime, where the chemical reaction causes the composition temperature to rise sharply. Once the temperature of the composition peaks, the composition enters a third, cooling regime, during which the temperature of the composition decreases back to room temperature.

The dotted line of FIG. 13 represents the behavior of the composition when it is further heated such as by an injector system, referred to as condition 2. In contrast to condition 1, four regimes of behavior are exhibited by the composition under condition 2. The first, low heating rate regime, the second, high heating rate regime, and the third, cooling regime, are again observed. In contrast with condition 1, however, a new, injector heating regime, is observed between the first and second regimes. This new regime exhibits a rapid increase in the composition temperature due to injector heating of the composition. Although the composition temperature is observed to peak and fall towards the end of the duration of this regime, the temperature does not fall back to the same level as observed under condition 1 at about the same time. Therefore, when the second, high heating rate regime is entered, the temperature of the composition under condition 2 is greater than that under condition 1 and the composition temperature rises to a peak temperature which is greater than that achieved under condition 1.

The setting time of the compositions under conditions 1 and 2 can be measured according to ASTM standard F451 and compared to identify changes in setting time between the two conditions. It is observed that the setting time of the composition under condition 1 is approximately 38 minutes, while the setting time of the composition under condition 2 is approximately 28 minutes, a reduction of about 10 minutes. Thus, by heating the bone cement, the setting time of embodiments of the bone cement composition may be reduced.

From the forgoing, then, it can be appreciated that by varying the BPO and/or DMPT concentrations of the bone cement composition and/or by heating the bone cement composition, the setting time of the bone cement may be increased or decreased. Furthermore, in certain embodiments, the concentration of BPO and/or DMPT in the bone cement may be varied and the composition may be heated so as to adjust the setting time to a selected value. As discussed above, in certain embodiments, the setting time is selected to be about 25 minutes or more, more preferably about 30 minutes or more, more preferably about 35 minutes or more, and even more preferably about 40 minutes or more.

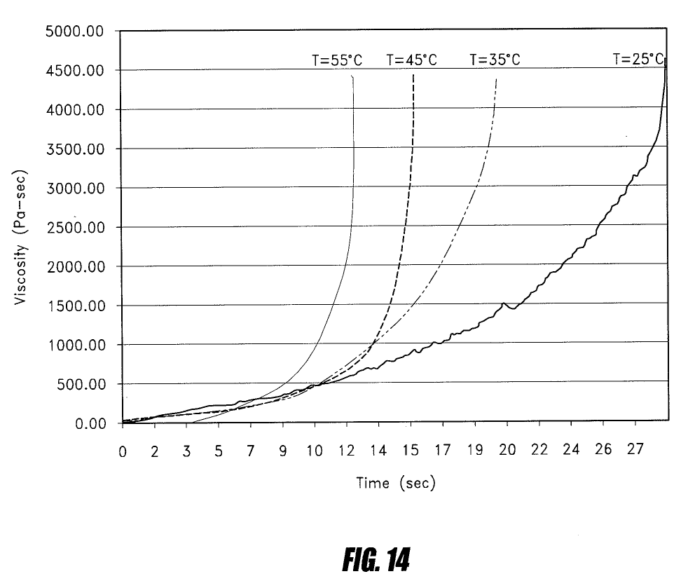

Embodiments of a bone cement composition may further be heated using the injector systems described herein in order to alter the viscosity of the composition. FIG. 14 illustrates measurements of viscosity as a function of time for an embodiment of the bone cement compositions heated to temperatures ranging between about 25° C. to 55° C. It may be observed that the bone cement at the lowest temperature, 25° C., exhibits the slowest rate of viscosity increase, while the bone cement at the highest temperature, 55° C., exhibits the highest rate of viscosity increase. Furthermore, at intermediate temperatures, the bone cement exhibits intermediate rates of viscosity increase.

From the behavior of condition 1 in FIG. 13, it can be seen that the peak temperature of the bone cement composition is higher when the cement is heated by the injector system. Furthermore, by adjusting the energy output of the injector system, the temperature to which the bone cement rises may be varied. Thus, embodiments of the injector system may be employed to deliver bone cements having selected levels of viscosity.

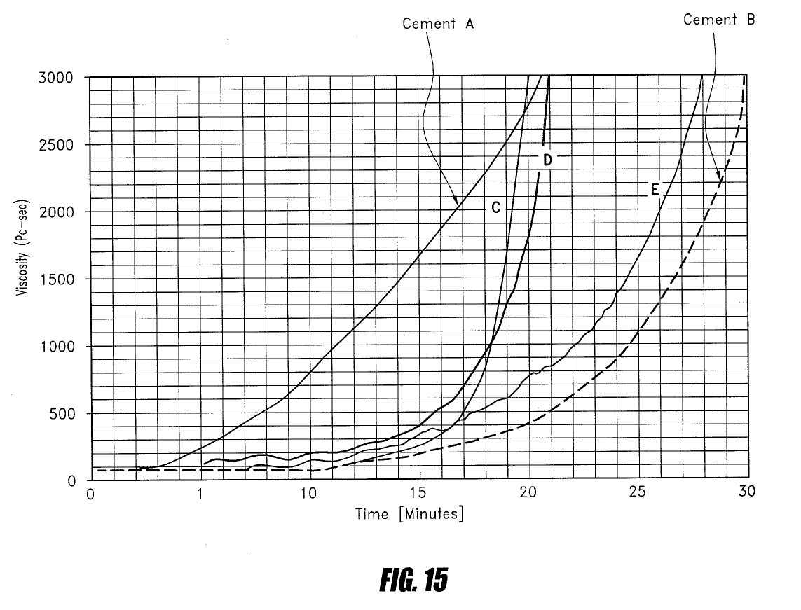

In one embodiment, a bone cement has a first component comprising greater than about 99 wt. % methyl methacrylate (MMA), and less than about 1 wt. % N,N-dimethyl-p-toluidine (DMPT), about 30 to 120 ppm hydroquinone on the basis of the total amount of the first component, and a second component comprising a powder component comprising less than 75 wt. % PMMA, less than 32 wt. % of an X-ray contrast medium; and less than 0.4 wt. % benzoyl peroxide (BPO) on the basis of the total weight of the second component. In another embodiment, the second component has less than 0.2 wt. % benzoyl peroxide (BPO) on the basis of the total weight of the second component, or less than 0.1 wt. % benzoyl peroxide (BPO) on the basis of the total weight of the second component. In such a formulation, the liquid weight/powder weight ratio is equal to or greater than about 0.4. In one embodiment indicated by Cement A in FIG. 15, the PMMA to monomer ratio is 2:1. In another embodiment indicated by Cement B in FIG. 15, the PMMA to monomer ratio is 2.5:1.

In another embodiment, a settable bone cement comprises mixable first and second components, wherein the first component comprises greater than about 99 wt. % methyl methacrylate (MMA), and less than about 1 wt. % N,N-dimethyl-p-toluidine (DMPT), about 30 to 120 ppm hydroquinone on the basis of the total amount of the first component, and wherein the second component comprises a PMMA component comprised of less than 75 wt. % PMMA, less than 32 wt. % of an X-ray contrast medium; and a selected wt. % of benzoyl peroxide (BPO) on the basis of the total weight of the second component. More particularly, the PMMA component includes first and second volumes of beads having first and second amounts of BPO, respectively. In one embodiment, the PMMA component includes a first volume of beads having greater than 0.4 wt. % BPO on the basis of the total weight of the PMMA component and the first volume has a mean bead size of less than 100 microns. In this embodiment, the PMMA component includes a second volume of beads having less than 0.4 wt. % BPO on the basis of the total weight of the PMMA component and the second volume has a mean bead size of greater than 100 microns. In another embodiment, the cement has a plurality of different PMMA beads sizes each carrying a different BPO amount, wherein the mean BPO amount among the plurality of beads is from 0.3 to 0.6% BPO on the basis of the total weight.

In another embodiment, the PMMA component includes a first volume of beads that has greater than 0.4 wt. % BPO on the basis of the total weight of the PMMA and the first volume has a mean bead size of greater than 100 microns. Further, the PMMA component includes a second volume of beads having less than 0.4 wt. % BPO on the basis of the total weight of the PMMA component and the second volume has a mean bead size of less than 100 microns.

In another method, the energy-delivery emitter 110 may be actuated to apply energy of at least about: 0.01 Watt, 0.05 Watt, 0.10 Watt, 0.50 Watt and 1.0 Watt. In another embodiment of a method, the applied energy may be modulated by a controller 145. In another embodiment of a method, the energy source and controller may be capable of accelerating the polymerization rate of the bone cement to the selected endpoint in less than 1 second, 5 seconds, 10 seconds, 20 seconds, 30 seconds, 45 seconds, 60 seconds and 2 minutes. In another embodiment of a method, the energy source and controller may be capable of accelerating the polymerization rate by at least 20%, 30%, 40%, 50%, 60%, 70%, 80%, 90% and 95% as compared to the polymerization rate absent the applied energy.

In another embodiment, a method of bone cement injection includes modulating a rate of bone cement flow in response to a determination of a selected parameter of the cement flow. Examples of the selected parameter may include the flow rate of the bone cement. A method of bone cement injection can further include applying thermal energy and modulating the thermal energy application from an emitter in the injector body to the cement flow. Some methods of bone cement injection can further include modulating the application of energy in response to signals that relate to a selected parameter such as flow rate of the cement flow.

Of particular interest, another embodiment of a method of bone cement injection comprises (a) providing a bone cement injector body carrying a PTCR (positive temperature coefficient of resistance) material in a flow channel therein, (b) applying a selected level of energy to a cement flow through the PTCR material, and (c) utilizing an algorithm that processes impedance values of the PTCR material to determine the cement flow rate. The method of bone cement injection may further include modulating a cement injection parameter in response to the processed impedance values. Examples of the cement injection parameter may include, but are not limited to flow rate, pressure, and power applied to the flow.

Another embodiment of a method of bone cement injection can include (a) providing a bone cement injector body carrying a PTCR material or other thermal energy emitter in a flow channel therein, (b) causing bone cement to flow through the flow channel at a selected cement flow rate by application of a selected level of energy delivery to the cement flow through the emitter, and (c) modulating the selected flow rate and/or energy delivery to maintain a substantially constant impedance value of the emitter material over a cement injection interval. The selected cement injection interval can include at least about 1 minute, at least about 5 minutes, at least about 10 minutes and at least about 15 minutes.

In another embodiment, a method can modulate the selected flow rate and/or energy delivery to maintain a substantially constant viscosity of bone cement ejected from the injector over a selected cement injection time interval. The time interval may include from about 1 minute to 10 minutes. The system and energy source can be configured for applying energy of at least 0.01 Watt, 0.05 Watt, 0.10 Watt, 0.50 Watt and 1.0 Watt. In another embodiment, the energy source and controller can be configured for accelerating polymerization rate of the bone cement to a selected endpoint in less than about: 1 second, 5 seconds, 10 seconds, 20 seconds, 30 seconds, 45 seconds, 60 seconds and 2 minutes.

Another embodiment of a method of bone cement injection may utilize embodiments of the systems 10 and 400 as describe above. Such methods may include (a) providing a bone cement injector body with a flow channel extending therethrough from a proximal handle end though a medial portion to a distal end portion having a flow outlet, (b) causing cement flow through the flow channel, and (c) warming the cement flow with an energy emitter in a proximal end or medial portion thereof to initiate or accelerate polymerization of the cement of the cement flow. The method may further include providing a flow rate of the cement flow that ranges from 0.1 cc/minute to 20 cc/minute, from about 0.2 cc/minute to 10 cc/minute and from about 0.5 cc/minute to 5 cc/minute.

Embodiment of the above-described method of bone cement injection can allow a predetermined cement flow rate to provide a selected interval in which the cement flows are allowed to polymerize in the flow channel downstream from the energy emitter. This method may include providing a selected interval of greater than about 1 second, greater than about 5 seconds, greater than about 10 seconds, greater than about 20 seconds, and greater than about 60 seconds.

The above-described method can utilize an energy emitter that applies energy sufficient to elevate the temperature of the bone cement by at least about 1° C., at least about 2° C. and at least about 5° C. The method of bone cement injection can include utilizing an energy emitter that applies at least about 0.1 Watt of energy to the cement flow, at least about 0.5 Watt of energy to the cement flow, and at least about 1.0 Watt of energy to the cement flow. The method can include the flow rate of the cement flow being adjusted in intervals by controller 145, or being continuously adjusted by a controller.

In another method, a bone cement injection system as described herein can utilize a controller 145 and algorithms for applying energy to bone cement flows to allow the bone cement 130 exiting the injector to possess a selected temperature that is higher than ambient temperature of the injector. This ability reflects the fact that polymerization has been accelerated, thus reducing the amount of total heat released into bone. More particularly, the method can include injecting a settable bone cement into bone after mixing a first component and a second component of the bone cement, thereby initiating a chemical reaction to initiate setting of the bone cement, accelerating the polymerization with applied energy from an external source, and ejecting the bone cement from an injector portion positioned in bone. The bone cement, upon ejection, may posses a temperature greater than ambient temperature of the injector. The method can include ejecting the bone cement from a terminal portion of an injector positioned in bone at a temperature of at least about: 28° C., 30° C., 32° C., 34° C., 36° C., 38° C., 40°, C 42° C., 44°C., 46° C., 48° C., 50° C., 52° C., 54° C., 56° C., 58° C., 60° C., 62° C., 64° C., 66° C., 68° C., 70° C., 72° C., 74° C., 76° C., 78° C. and 80° C.

In another embodiment, a method of injecting a bone cement into bone can include mixing first and second bone cement components thereby causing an exothermic chemical reaction which results in a thermal energy release, and then actuating an injector control system capable of controlling the temperature of the bone cement before the bone cement contacts bone. In general, the actuating step can include (i) controlling the flow rate of the bone cement within a flow passageway of an injector system, (ii) controlling the application of energy to the bone cement from an emitter operatively coupled to an energy source, and (iii) controlling the driving force applied to the flow of bone cement which may benefit from adjustment based on the bone cement viscosity.

The actuating step can also include sensing an operating parameter of the bone cement flow to which the controller is responsive. The operating parameter can include the bone cement flow rate, the bone cement temperature, the driving force applied to the cement flow, the energy applied to the cement from an emitter coupled to an energy source and cement viscosity and environmental conditions, such as temperature and humidity in the environment ambient to the injector system. Thus, the controller 145 can be capable of modulating the flow rate, modulating the applied energy and/or modulating the driving force in response to sensing any one or more of the above operating parameters.

In another embodiment, a method of injecting a bone cement can include mixing the first and second bone cement components so as to cause an exothermic chemical reaction that results in a thermal energy release, and actuating an injector control system which is capable of controlling the amount of thermal energy released from the cement before the bone cement contacts bone tissue to thereby reduce the thermal energy released into the bone.

The thermal energy released from the cement can be directly related to the level of polymerization acceleration from the applied energy as well as the dwell time of the cement within the flow channel before the cement exits the outlet in a terminal portion of the injector. The dwell time of the cement in the flow channel can be controlled by controller 145 as described above, where at least one of the flow rate and driving force applied to the cement flow can be modulated. In the system embodied in FIGS. 1-6, the application of energy by emitter 110 in component 105 can provide for a dwell time within the flow channel 112 before exiting outlet 122 for a flow interval of at least about: 5 seconds, 10 seconds, 20 seconds, 30 seconds, 40 seconds and 60 seconds. This method of conditioning and injecting bone cement can allow a thermal energy release from the bone cement before the bone cement contacts bone of at least about 10%, at least about 15%, at least about 20%, at least about 25%, at least about 30%, at least about 35%, at least about 40%, at least about 45% and at least about 50%.

In another embodiment, it can be understood that the systems and methods disclosed herein may be further employed in order to control the amount of thermal energy released from the bone cement before the cement contacts bone tissue to thus reduce the amount of thermal energy released into the bone.

For example, in one embodiment, a method of injecting a bone cement can control the amount of thermal energy released by the bone cement before the cement contacts bone tissue. The method includes controlling an injector control system that is capable of controlling the rate of chemical reaction before the bone cement contacts bone tissue. The reaction rate can be adjusted by the controller such that the maximum composition temperature is reached when the cement is within the flow channel of the injector system, prior to reaching the bone tissue. Beneficially, in this manner, the amount of total thermal energy released by the bone cement is released while the bone cement is still within the flow channel of the injector system, before the bone cement contacts the bone tissue. This method substantially reduces the amount of thermal energy which is released by the bone cement into the bone tissue.

In another method of injecting bone cement, the actuating step can include allowing at least about 10% of the total thermal energy released from a bone cement to be release while the bone cement flows within the injector system. In certain embodiments, such energy release may be accomplished by providing a mean cement flow rate of at least about 0.1 cc/min, at least about 0.5 cc/min, at least about 1.0 cc/min, at least about 1.5 cc/min, at least about 2.0 cc/min and at least about 2.5 cc/min during heating within the bone cement injector. The method may further include maintaining the bone cement within the cannula for at least about 20 seconds after being heated.

In another method, the actuating step can allow at least about 10% of the total thermal energy released from a bone cement to flow over a flow distance within the flow channel 112 of the injector system of at least about 5 mm, at least about 10 mm, at least about 20 mm, at least about 30 mm, at least about 40 mm, at least about 50 mm, at least about 60 mm, at least about 70 mm, at least about 80 mm, at least about 90 mm and at least about 100 mm.

In certain embodiments, the methods described above can apply energy to a selected volume of a bone cement mixture. A selected amount of thermal energy from the exothermic reaction of the bone cement components may be released within the flow channel so as to inhibit a selected portion of the thermal energy from reaching a patient's bones. Beneficially, in this manner, a reduction in the thermal effects in the bone due to introduction of the bone cement within the bone may be achieved. Embodiments of the method can include selecting first and second bone cement components, or precursors, that result in a peak temperature of the bone cement composition during curing of less than about: 75° C., 70° C., 65° C. and 60° C. Embodiments of such bone cements may include those bone cements described herein. In certain embodiments, the injected volume subjected to the accelerated chemical reaction releases less thermal energy than a cement mixture not subjected to the accelerated chemical reaction, wherein the release is at least about: 10%, 20%, 30%, 40% and 50% less thermal energy release than a cement mixture not subjected to the accelerated chemical reaction.

Thus, from the above disclosure, it can be understood that some embodiments of bone cement injection systems can include first and second bone cement components, or precursors, that, upon mixing, result in a chemical reaction that sets the cement mixture. The bone cement injection system can further include an injector system that may include a drive system for inducing flow of the cement mixture through the system and into bone. The bone cement injection system can further include an energy emitter for applying energy to the cement mixture in the injector system to thereby accelerate the chemical reaction between the first and second bone cement components therein. The bone cement injection system can further include a controller operatively coupled to at least one of the drive system and energy emitter, for controlling the acceleration of the chemical reaction in the bone cement. In one embodiment, the first and second bone cement components or precursors post-mixing can have a peak temperature of less than about: 75° C., 70° C., 65° C. and 60° C. The drive system and controller may further be capable of controllably applying a driving force to the cement mixture in the injector system of at least about: 500 psi, 1,000 psi, 1,500 psi, 2,000 psi, 2,500 psi, 3,000 psi, 3,500 psi, 4,000 psi, 4,500 psi and 5,000 psi.

In one embodiment, the drive system and controller can be capable of controllably maintaining a substantially constant flow rate of the cement mixture. Examples of the flow rate control may include, but are not limited to, flow rate variations that are within less than about: 1% variation; 5% variation; 10% variation and 15% variation.

In one embodiment, the drive system and controller can be capable of controlling a mean cement mixture flow rate. The mean cement flow rate may include at least about 0.1 cc/min, at least about 0.5 cc/min, at least about 1.0 cc/min, at least about 1.5 cc/min, at least about 2.0 cc/min and at least about 2.5 cc/min. The energy emitter and controller may further be capable of controllably applying energy to the cement mixture. In certain embodiments the controller may provide at least about: 20 joules/cc, 40 joules/cc, 60 joules/cc, 80 joules/cc, 100 joules/cc and 120 joules/cc, 140 joules/cc, 160 joules/cc and 180 joules/cc.

In certain embodiments, a bone cement injection system can include an energy emitter and controller capable of providing a dynamic or a pre-programmed adjustment of applied energy to the cement mixture in response to a signal indicative of the flow rate of the cement mixture. The signal can be a feedback signal to the controller 145 indicative of at least one of the temperature of the cement mixture, the viscosity of the cement mixture, the flow rate of the cement mixture and the driving force applied to the cement mixture, at least one environmental condition and combinations thereof.

Now returning to FIG. 15, embodiments of bone cement described above can be characterized by time-viscosity curves of cement A and cement B, with commercially available cements indicated at C, D and E (in FIG. 15, C is Mendec Spine bone cement; D is DePuy Vertebroplastic cement; E is Arthrocare Parallax acrylic resin). Cement A includes a first monomer-carrying component and a second polymer-carrying component, wherein post-mixing the mixture is characterized by a time-viscosity curve slope of less than 200 Pa·s/minute until the mixture achieves a viscosity of 3000 Pa·s. Another cement embodiment includes a first monomer-carrying component and a second polymer-carrying component, wherein post-mixing the mixture is characterized by a time-viscosity curve slope of less than 200 Pa·s/minute until to the mixture achieves a viscosity of 2500 Pa·s. Alternatively, the bone cement B includes a first monomer-carrying component and a second polymer-carrying component, wherein post-mixing the mixture is characterized by a time-viscosity curve slope of less than 200 Pa·s/minute for at least 20 minutes, 25 minutes and 30 minutes.

In one embodiment, a bone cement may include a first monomer-carrying component and a second polymer-carrying component, wherein the mixture is characterized by having a viscosity of less than 500 Pa·s at 18 minutes post-mixing. The bone cement further can be characterized as having a time-viscosity curve slope of less than 200 Pa·s/minute for at least 5 minutes after achieving a viscosity of 500 Pa·s. The bone cement further can be characterized by a post-mixing time-viscosity curve slope of less than 100 Pa·s/minute for at least about 15 minutes, 16 minutes, 17 minutes, 18 minutes, 19 minutes and 20 minutes.

In one embodiment, a bone cement may include a first monomer-carrying component and a second polymer-carrying component, wherein post-mixing the mixture is characterized by a time-viscosity curve slope of less than 100 Pa·s/minute until to the mixture achieving a viscosity of 500 Pa·s. The bone cement post-mixing can be characterized by a time-viscosity curve slope of less than 100 Pa·s/minute immediately before the mixture achieves a viscosity of 800 Pa·s. The bone cement further can be characterized by a time-viscosity curve slope of less than 100 Pa·s/minute immediately before the mixture achieves a viscosity of 1000 Pa·s. The bone cement further can be characterized by a time-viscosity curve slope of less than 100 Pa·s/minute immediately before the mixture achieves a viscosity of 1500 Pa·s. The bone cement further can be characterized by a time-viscosity curve slope of less than 200 Pa·s/minute immediately before the mixture achieves a viscosity of 500 Pa·s. The bone cement further can be characterized by a time-viscosity curve slope of less than 200 Pa·s/minute immediately before the mixture achieves a viscosity of 1000 Pa·s. The bone cement further can be characterized by a time-viscosity curve slope of less than 200 Pa·s/minute immediately before the mixture achieves a viscosity of 1500 Pa·s. The bone cement further can be characterized by a time-viscosity curve slope of less than 200 Pa·s/minute immediately before the mixture achieves a viscosity of 2000 Pa·s.

In one embodiment, a bone cement may include a first monomer-carrying component and a second polymer-carrying component, wherein post-mixing the mixture is characterized by a time-viscosity curve having a rate of change of less than 20% or less that 40% over an interval of at least about: 5 minutes, 10 minutes, 15 minutes and 20 minutes.

In one embodiment, a bone cement can include a first monomer-carrying component and a second polymer-carrying component, wherein post-mixing the mixture of the first and second components is characterized as having a viscosity of less than about 100 Pa·s at 10 minutes post-mixing, or less than about 200 Pa·s at 15 minutes post-mixing, or less than about 500 Pa·s at 18 minutes post-mixing.

In one embodiment, a bone cement can include a first monomer-carrying component and a second polymer-carrying component, wherein post-mixing the mixture is configured to receive applied energy of at least about: 20 joules/cc, 40 joules/cc, 60 joules/cc, 80 joules/cc, 100 joules/cc and 120 joules/cc, 140 joules/cc, 160 joules/cc and 180 joules/cc without substantially setting in an interval of less than 10 minutes. The bone cement post-mixing mixture upon application of energy from an external source of at least 60 joules/cc is characterized as having a viscosity of greater than 500 Pa·s within about: 10 seconds, 30 seconds 60 seconds, 90 seconds, 120 seconds, 180 seconds and 240 seconds.

In another embodiment, a bone cement formulation described above may include first and second cement precursors, wherein the mixture is characterized by a post-mixing interval in which viscosity is between about 500 Pa·s and 5000 Pa·s, and in which the change of viscosity of less than 30%/minute. In another embodiment, the settable bone cement may include first and second cement precursors, wherein the mixture is characterized by a post-mixing interval in which viscosity is between about 500 Pa·s and 2000 Pa·s, and in which the change of viscosity of less than 20%/minute.

In another aspect, a settable bone cement can include a first monomer-carrying component and a second polymer-carrying component, wherein post-mixing the mixture is characterized by a change of viscosity of less than 20%/minute for at least three minutes after reaching about: 500 Pa·s, 1000 Pa·s, 1500 Pa·s and 2000 Pa·s. In another embodiment, the cement can include a first monomer-carrying component and a second polymer-carrying component, wherein post-mixing the mixture is characterized by a change of viscosity of less than 30%/minute for at least three minutes after reaching about: 500 Pa·s, 1000 Pa·s, 1500 Pa·s and 2000 Pa·s. In a related embodiment, the cement can include a first monomer-carrying component and a second polymer-carrying component, wherein post-mixing the mixture is characterized by a change of viscosity of less than 40%/minute for at least three minutes after reaching about: 500 Pa·s, 1000 Pa·s, 1500 Pa·s and 2000 Pa·s. In a related embodiment, the cement can include a first monomer-carrying component and a second polymer-carrying component, wherein post-mixing the mixture is characterized by a change of viscosity of less than 30%/minute for at least five minutes after reaching about: 1000 Pa·s, 1500 Pa·s, 2000 Pa·s, 2500 Pa·s, 3000 Pa·s, 3500 Pa·s and 4000 Pa·s. In another related embodiment, the cement can include a first monomer-carrying component and a second polymer-carrying component, wherein post-mixing the mixture is characterized by a change of viscosity of less than 40%/minute for at least five minutes after reaching about: 1000 Pa·s, 1500 Pa·s, 2000 Pa·s, 2500 Pa·s, 3000 Pa·s, 3500 Pa·s and 4000 Pa·s. In a related embodiment, the cement may include a first monomer-carrying component and a second polymer-carrying component, wherein post-mixing the mixture is characterized by a change of viscosity of less than 50%/minute for at least five minutes after reaching about: 1000 Pa·s, 1500 Pa·s, 2000 Pa·s, 2500 Pa·s, 3000 Pa·s, 3500 Pa·s and 4000 Pa·s.

In another aspect, a cement may include a first monomer-carrying component and a second polymer-carrying component, wherein the mixture is characterized by a rate of change of viscosity of less than about 50%/minute after achieving a viscosity of 5000 Pa·s. In a related embodiment, a cement may include a first monomer-carrying component and a second polymer-carrying component, wherein the mixture is characterized by a rate of change of viscosity of less than about 50%/minute after achieving a viscosity of 4000 Pa·s. In a related aspect, a cement may include a first monomer-carrying component and a second polymer-carrying component, wherein the mixture is characterized by a rate of change of viscosity of less than about 50%/minute after achieving a viscosity of 3000 Pa·s.

In another aspect, a cement can include a first monomer-carrying component and a second polymer-carrying component, wherein post-mixing the mixture is characterized by a rate of change of viscosity of less than 50%/minute for an interval preceding the point in time the mixture achieves 5000 Pa·s, the interval being at least about: 2 minutes, 3 minutes, 4 minutes, 5 minutes, 6 minutes and 8 minutes. In a related aspect, a cement can include a first monomer-carrying component and a second polymer-carrying component, wherein post-mixing the mixture is characterized by a rate of change of viscosity of less than 40%/minute for an interval preceding the point in time the mixture achieves 5000 Pa·s, the interval being at least about: 2 minutes, 3 minutes, 4 minutes, 5 minutes, 6 minutes and 8 minutes. In a related aspect, a cement can include a first monomer-carrying component and a second polymer-carrying component, wherein post-mixing the mixture is characterized by a rate of change of viscosity of less than 30%/minute for an interval preceding the point in time the mixture achieves 5000 Pa·s, the interval being at least about: 2 minutes, 3 minutes, 4 minutes, 5 minutes, 6 minutes and 8 minutes.

In another aspect, a cement can include a first monomer-carrying component and a second polymer-carrying component, wherein the mixture is characterized by a post-mixing interval of at least about: 4 minutes, 6 minutes, 8 minutes or 10 minutes in the interval preceding the point in time the mixture achieves 3000 Pa·s. In a related aspect, a cement may include a first monomer-carrying component and a second polymer-carrying component, wherein the mixture is characterized by a post-mixing interval of at least about: 4 minutes, 6 minutes, 8 minutes or 10 minutes in the interval preceding the point in time the mixture achieves 4000 Pa·s. In a related aspect, a cement can include a first monomer-carrying component and a second polymer-carrying component, wherein the mixture is characterized by a post-mixing interval of at least about: 4 minutes, 6 minutes, 8 minutes or 10 minutes in the interval preceding the point in time the mixture achieves 5000 Pa·s.

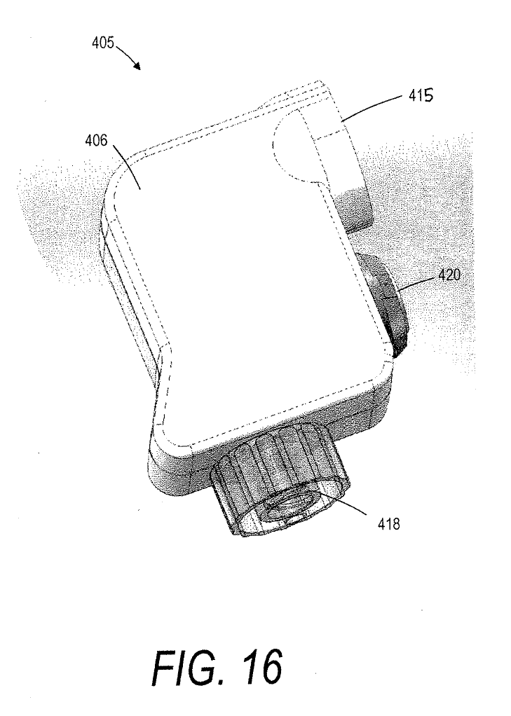

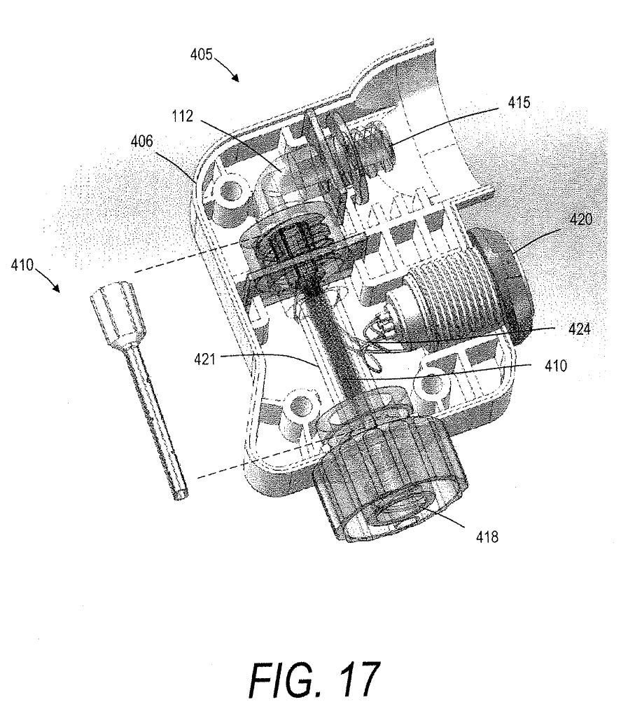

Referring now to FIGS. 16-17, a handle portion 405 (or cement activation component) of a bone cement injector similar to component 105 of FIGS. 1-2 is shown. The handle portion 405 has a housing 406 that houses a thermal energy emitter 410 (FIG. 17) similar to the emitter 110 in FIGS. 1-4 above that can be operated to apply energy to a bone cement flow through a flow channel 112 in the system (FIG. 17). The handle portion 405 can be made of plastic or any other suitable material. As similarly described previously with respect to FIGS. 1-4, the handle portion 405 can include a first connector 415 for detachably coupling a source of a bone cement inflow to the handle portion and flow channel 112 which extends therethrough. As shown in the illustrated embodiment, the handle portion 405 can also have a second connector 418 for detachably coupling the handle portion 405 and flow channel 112 to an injector cannula or needle for delivering bone cement into a bone (cf. FIG. 1-2). In one embodiment, the cannula can be used for injecting bone cement into the interior of a vertebra, but it may also be used for injecting bone cement into different bones within a body. An electrical connector 420 in the handle portion can be detachably connected to a power cable for coupling the emitter 410 with a power source. The handle portion 405 may be constructed of different materials including but not limited to composites and polymers.

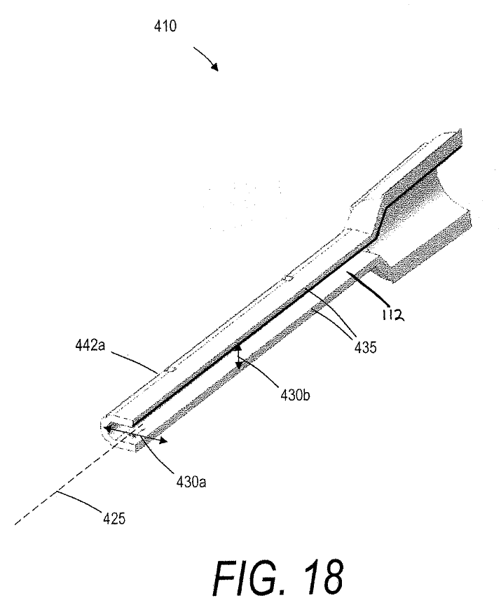

As can be seen in FIGS. 17-18, the emitter or energy applicator 410 is disposed in a fixed position in the interior of the handle portion 405 by any suitable mechanism, such as molded webs elements within mating sides of a plastic housing 421. In the embodiment of FIG. 17, the emitter 410 is coupled to electrical leads 424 which extend to the connector 420. In one embodiment, the emitter is used for applying energy to any polymer and monomer mixture, such as a PMMA mixture described above.

Referring now to FIG. 18, a longitudinal cross-sectional view of the emitter 410 detached from the handle portion 405 shows the component having an axis 425 with an axial flow channel 112 extending therethrough. In one embodiment, the emitter or energy applicator component 410 can be at least partly a polymer that has an axial length of at least 1 mm, 5 mm, 10 mm, 20 mm, and 40 mm. By way of example only, emitter 410 can be a resistive heater of a resistive metal, a resistive metal-polymer composite, or a positive temperature coefficient of resistance (PTCR) polymer. In such embodiments, an electrical source can be coupled to the component for heating thereof. In another embodiment, the emitter component 410 can comprise a polymer that itself may be transparent or translucent (and electrically insulative) to cooperate with a light source that is configured to apply energy to bone cement. In still other embodiments, the emitter component 410 may be at least partly a metal with an axial length of at least 1 mm, 5 mm, 10 mm, 20 mm, and 40 mm, and operatively coupled to an ultrasound source, a microwave source etc. for applying energy to a bone cement flow.

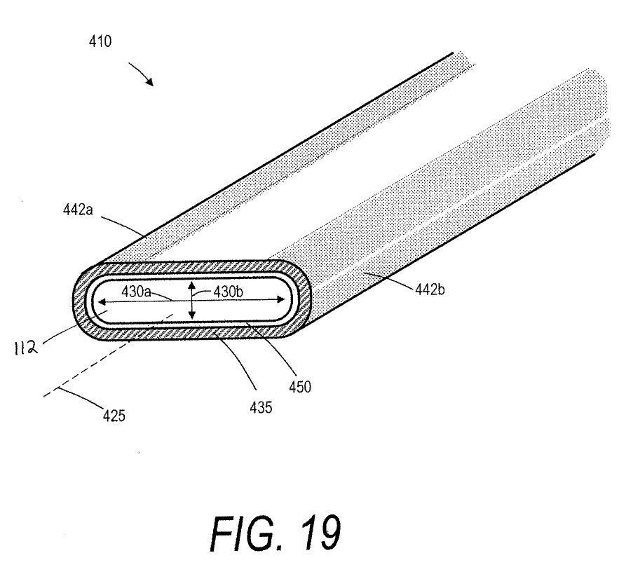

In one embodiment as shown in FIGS. 18-19, at least a portion of the flow channel 112 has a non-round cross-section that is characterized by a major and minor axis, 430a and 430b, respectively. The major axis 430a is greater than the minor axis 430b by at least 110%, 150%, and 200%. In still other embodiments, the non-round cross-section may be oval or rectangular in shape, or star shaped or any other shape to reduce the cross section of the bone cement flow to allow rapid and uniform heating of the cement by thermal diffusion of heat through the cement from contact with the wall 435 of emitter 410.

In one embodiment, at least one electrode portion of the emitter 410 is coupled to electrical leads 424. In FIGS. 18 and 19 it can be seen that first and second opposing polarity electrodes 442a and 442b are disposed on opposite sides of the emitter, spaced apart by the major axis 430a. It has been found that such an electrode arrangement provides uniform heating of the walls 435 of the emitter. In other embodiments, the first and second opposing polarity electrodes may extend circumferentially, axially, or angularly about the axis of the component. In still other embodiments the opposing polarity electrodes may be positioned about opposing sides of either the major or minor axis of the non-round flow channel. In yet another embodiment, the at least one energy emitter includes but is not limited to a resistive heater, a light source, or an ultrasound source. The resistive heat source, the light source, or the ultrasound source can be disposed on at least one side of the major or minor axis of the non-round flow channel. In still other embodiments, the resistive heat source, the light source, or the ultrasound source can be disposed axially, circumferentially, or angularly about the major or minor axis of the flow channel.

Referring to FIG. 19, an emitter 410 is shown for applying energy to a bone cement having an axis 425 with an axial flow channel 112 extending therethrough, and an interior surface layer 450 of the flow channel 112 that comprises a material that limits bone cement flow turbulence and/or increases laminar flow. The surface layer 450 can be made of a material having a static coefficient of friction of less than 0.5. In still other embodiments the material has a static coefficient of friction of less than 0.2 or less than 0.1. In another embodiment the surface of the material selected has a wetting contact angle greater than 70 degrees. In still other embodiments, the wetting contact angle can be greater than 80, 90, 100, or 110 degrees. In another embodiment, the surface of the material selected also may have an adhesive energy of less than 100 dynes/cm. In still other embodiments, the surface of the material selected may have an adhesive energy of less then 75 dynes/cm, or less than 50 dynes/cm.

In another embodiment the material used for the interior surface layer 450 of the flow channel 112 may be a polymer or ceramic. In still other embodiments, the material used for the interior surface of the flow channel may be selected from the group comprising of Polytetrafluoroethylene (PTFE), Perfluoroalkoxy (PFA), Fluorinatedethylenepropylene (FEP), Ethylenechlorotrifluoroethylene (ECTFE), ETFE, Polyethylene, Polyamide, PVDF, Polyvinyl chloride, and silicone. In still another embodiment, the material may by ultrahydrophobic, hydrophilic, oleophobic, or oleophilic.

The above description is intended to be illustrative and not exhaustive. Particular characteristics, features, dimensions and the like that are presented in dependent claims can be combined and fall within the scope of the invention. The disclosure also encompasses embodiments as if dependent claims were alternatively written in a multiple dependent claim format with reference to other independent claims. Specific characteristics and features of the systems and methods are described in relation to some figures and not in others, and this is for convenience only. While the principles of the invention have been made clear in the exemplary descriptions and combinations, it will be obvious to those skilled in the art that modifications may be utilized in the practice of the invention, and otherwise, which are particularly adapted to specific environments and operative requirements without departing from the principles of the invention. The appended claims are intended to cover and embrace any and all such modifications, with the limits only of the true purview, spirit and scope of the invention.

Of course, the foregoing description is that of certain features, aspects and advantages of the present invention, to which various changes and modifications can be made without departing from the spirit and scope of the present invention. Moreover, the bone treatment systems and methods need not feature all of the objects, advantages, features and aspects discussed above. Thus, for example, those of skill in the art will recognize that the invention can be embodied or carried out in a manner that achieves or optimizes one advantage or a group of advantages as taught herein without necessarily achieving other objects or advantages as may be taught or suggested herein. In addition, while a number of variations of the invention have been shown and described in detail, other modifications and methods of use, which are within the scope of this invention, will be readily apparent to those of skill in the art based upon this disclosure. It is contemplated that various combinations or subcombinations of these specific features and aspects of embodiments may be made and still fall within the scope of the invention. Accordingly, it should be understood that various features and aspects of the disclosed embodiments can be combined with or substituted for one another in order to form varying modes of the discussed bone treatment systems and methods.

What is claimed is:

1. A medical device for applying energy to a bone cement comprising:
a member having a flow channel extending therethrough, wherein at least a portion of the flow channel has a non-round cross section;
at least one energy emitter operatively coupled to the member and configured to apply energy to a bone cement flowing through the flow channel; and
a bone cement source coupleable to the flow channel.

2. The medical device of claim 1, wherein an interior surface of the flow channel comprises a material that limits cement flow turbulence.

3. The medical device of claim 1, wherein the at least one energy emitter comprises at least one electrode.

4. The medical device of claim 1, wherein the at least one energy emitter comprises at least first and second opposing polarity electrodes.

5. The medical device of claim 1, wherein the at least one energy emitter is selected from the group comprising a resistive heater, a light source, an LED, a microwave source and an ultrasound source.

6. The medical device of claim 1, wherein the member is made at least partly of a polymer.

7. The medical device of claim 6, wherein the polymer is transparent.

8. The medical device of claim 6, wherein the polymer is electrically conductive.

9. The medical device of claim 6, wherein the polymer has a positive temperature coefficient of resistance.

10. The medical device of claim 1, wherein the member is at least partly a metal.

11. The medical device of claim 4, wherein the opposing polarity electrodes extend axially relative to an axis of the flow channel.

12. The medical device of claim 4, wherein the opposing polarity electrodes extend circumferentially or angularly relative to an axis of the flow channel.

13. The medical device of claim 4, wherein the opposing polarity electrodes comprise at least part of a wall of the member defining the flow channel.

14. The medical device of claim 1, wherein the non-round cross-section is characterized by a major and minor axis, further comprising opposing polarity electrodes about opposing sides of the major axis.

15. The medical device of claim 1, wherein the non-round cross-section is characterized by a major and minor axis, further comprising an energy emitter disposed on at least one side of the minor axis, the energy emitter selected from the group comprising a resistive heater, a light source, and LED, a microwave source and an ultrasound source.

16. The medical device of claim 1, wherein the non-round cross-section has a major axis and a minor axis, and wherein the major axis is greater than the minor axis by at least 200%.

17. The medical device of claim 1, wherein the member has an axial length of at least 1 mm.

18. The medical device of claim 1, further comprising a cannula coupleable to the flow channel, at least a portion of the cannula being introducible into a bone and configured to direct the flow of bone cement into the bone.

19. A medical device for applying energy to a bone cement comprising:
a member with a flow channel extending therethrough;
a bone cement source coupleable to the flow channel; and
at least one energy emitter operatively coupled to the member and configured to apply energy to bone cement in the flow channel;
wherein an interior surface of the flow channel comprises a material that limits cement flow turbulence.

20. The device of claim 19, wherein at least a portion of the flow channel has a non-round cross section.

21. The device of claim 20, wherein the portion of the flow channel having a non-round cross section further comprises the material that limits cement flow turbulence.

22. The device of claim 19, wherein the material increases laminar flow.

23. The device of claim 19, wherein the material has a static coefficient of friction of less than 0.5.

24. The device of claim 19, wherein the material is selected from the group comprising PTFE (Polytetrafluoroethylene), PFA (Perfluoroalkoxy), FEP (Fluorinatedethylenepropylene), ECTFE (Ethylenechlorotrifluoroethylene), ETFE, Polyethylene, Polyamide, PVDF, Polyvinyl chloride and silicone.

25. The device of claim 19, wherein the material is a polymer.

26. The device of claim 19, wherein the material is a ceramic.

27. The device of claim 19, wherein the material is ultrahydrophobic, hydrophilic, oleophobic, and oleophilic.

28. The device of claim 19, wherein a surface of the material has a wetting contact angle greater than 70°.

29. The device of claim 19, wherein a surface of the material surface has an adhesive energy of less than 100 dynes/cm.

30. The device of claim 19, wherein an energy emitter is selected from the group comprising a resistive heater, at least one electrode coupled to an electrical source, a light source, an LED, an ultrasound waveguide and microwave antenna.
